(19) United States
(12) Patent Application Publication
(10) Pub. No.: US 2010/0152418 A1
(43) Pub. Date: Jun. 17, 2010

(54) Switch-Peptides as Tool for the Study of Fibrillogenesis

(75) Inventors: Manfred Mutter, Belmont-Lausanne (CH); Gabriele Tuchscherer, Belmont-Lausanne (CH)

Correspondence Address: HARNESS, DICKEY & PIERCE, P.L.C., P.O. BOX 8910, RESTON, VA, 20195, US

(73) Assignee: EPFL ECOLE POLYTECHNIQUE FEDERALE DE LAUSANNE, Lausanne (CH)

(21) Appl. No.: 11/922,207

(22) PCT Filed: Jun. 16, 2006
(86) PCT No.: PCT/IB2006/001617

§ 371 Date: Dec. 14, 2007

Related U.S. Application Data
(60) Provisional application No. 60/691,278, filed on Jun. 17, 2005.

(51) Int. Cl. C07K 2/00; C12P 21/00

(52) U.S. Cl. 530/300; 435/68.1

(57) ABSTRACT

The present invention relates to a method for the manufacture of a peptidic folding precursor (Switch-Peptide) stable and soluble at physiological conditions, derived from a peptide having a potential for self-assembling and fibrillogenesis. Another object of the invention is to provide a tool for the quantitative, controlled in vitro study of fibrillogenesis and its inhibition of peptides involved in degenerative diseases.

### FIELD OF THE INVENTION

The present invention relates to a method for the manufacture of a stable and soluble peptidic folding precursor (Switch-Peptide) at physiological conditions, derived from a peptide having a potential for self-assembling and fibrillogenesis. Another object of the invention is to provide a tool for the quantitative, controlled in vitro study of fibrillogenesis and its inhibition of peptides/proteins involved in degenerative diseases.

### BACKGROUND OF THE INVENTION

Alzheimer's disease (AD) is a very common form of degenerative disorder, involving memory loss, confusion, and various cognitive disabilities. AD was found to be essentially associated with the appearance of amyloid deposits (e.g. in the form of fibril-containing plaques, and the like), which are typically insoluble fibrillar aggregates that have a common structural motif: the beta-pleated sheet conformation. Said amyloid deposits generally comprise aggregates of a 39-42 residue peptide called amyloid-beta peptide (amyloid-β, A β). Such amyloid-beta aggregates are believed to play an important role in the early pathogenesis of AD. Therefore, amyloid deposits and fibrillogenesis are considered as an attractive target in treating AD.

Amyloid formation relates to protein folding and conformation, wherein initially mainly random-coil soluble peptides turn into essentially insoluble aggregates by adopting a beta-pleated sheet conformation.

The onset of conformational transitions as origin of peptide self-assembly is considered as a fundamental molecular event in early processes relevant in degenerative diseases [1-4]. A detailed investigation of these processes is hampered by intrinsic problems such as the high tendency of the involved peptides for β-sheet formation and spontaneous aggregation, limiting their experimental accessibility [5-7]. Applicant has recently developed a new generation of switch-peptides [8] allowing for the induction of conformational transitions using intramolecular O→N acyl migrations in situ [9]. The well-established O→N acyl transfer reaction as key step in protein splicing has recently found considerable attention [11]. For example, pH-induced acyl transfer reactions have been applied in prodrug design [12] and as solubilizing technique in the solid-phase synthesis and HPLC purification of difficult peptide sequences, including amyloid β (1-42) [13, 14].

For potential applications in vitro and in vivo, Applicant now explores the sequential triggering of O→N-acyl migrations in amyloid β (A β) derived switch-peptides as a tool for studying onset and inhibition in polypeptide folding, self-assembly and aggregation.

Aggregation of Aβ has been modelled in vitro under physiological conditions by the deposition of synthetic radio labelled Aβ onto AD amyloid in unfixed preparations (slide mounted sections) of AD human cerebral cortex²¹. This model system has been used to understand the key mechanistic and conformational features of Aβ aggregation, and works well for the characterization of agents that affect aggregation rates²². However, the requirements for human tissue and centrifugation make the assay troublesome for high throughput screening.

Esler and coworkers showed that synthetic fibrillar Aβ aggregates, immobilized on a polymer matrix, which they called as “synthaloid” can serve as a template for Aβ deposition that closely resembles Aβ deposition onto plaques in Aβ brain tissue. This system is suitable for highthroughput screening for the identification of Aβ aggregation inhibitors under physiological conditions.

Mark A. Findeis et al. ²³ have developed an assay to evaluate potential inhibitors of Aβ based on agitating a solution of synthetic Aβ 1-40 and monitoring the polymerization kinetics.

Most reports for monitoring fibril formation and screening inhibitors of fibril formation are based on full length Aβ 1-40, Aβ 1-42 or amylin peptide. Due to uncontrolled self-association and subsequent aggregation, these methods become cumbersome. Moreover, the state of aggregation at t=0 is never well defined.

The present invention provides a novel strategy to by-pass most of these problems, using ‘host-guest switch-peptides’ to monitor fibril formation and thereby screening the inhibitory effect of β breaker molecules. HQKLVFFAED and NFGAIL, the partial sequences of Aβ (Aβ 14-24) and amylin (amylin 17-22) peptide, respectively, are known to play a key role in the nucleation of their aggregation. However, these small peptide fragments do not show β-sheet in aqueous solution as monitored by CD. On the other hand, oligopeptides of alternative hydrophobic and hydrophilic amino acid, for example (Leu-Ser)ₙ, exhibit high potential for (3-sheet formation. To mimic the aggregation phenomena of native AD and amylin peptide, a host-guest peptide is designed, inserting HQKLVFFAED and NFGAIL in Leu-Ser oligomers. This host-guest peptide indeed forms (3-sheet structure in aqueous solution leading to amyloid-like aggregation. To properly initiate the conformational transition from a flexible random coil conformation, a serine switch is introduced in the middle of these sequences, replacing Ala²¹ of Aβ and GlY¹⁹ of amylin, thus providing host-guest switch-peptides (HGSP). By triggering the acyl migration upon changing the pH, these peptides are designed to convert, from a non functional (S_{off}) to a functional (Sₒₙ) state, as schematically shown in FIG. 15. The rate of conversion can be controlled by changing pH, temperature and solvent system. Incubating the (β-breaker molecules with these HGSP, prior to the triggering of the switch, should influence the kinetics of the conformational transition, hence the aggregation of the HGSP. By comparing the kinetics of aggregation of HGSP in the presence or absence of the β-breaker molecules, one can determine the efficiency of the (β-breaker compound. FIG. 15 represents the schematic diagram of the β-breaker or inhibitor screening system, developed in this way.

It is an object of the present invention to provide peptidic folding precursors (Switch-Peptide) at physiological conditions (pH 7.0 and temperature of 37° C.), derived from a peptide having a potential for self-assembling and fibrillogenesis.

It is a further object of the present invention to provide compounds which are easily obtainable via standard preparation techniques. More particularly, it is an object of the present invention to provide peptide-derived compounds which are readily stable and soluble at physiologic conditions (pH and temperature).

Another object of the present invention is to provide a tool for the quantitative, controlled in vitro study of fibrillogenesis and its inhibition of peptides/proteins involved in degenerative diseases.

### SUMMARY OF THE INVENTION

This and further objects have been achieved with a novel method for the manufacture of a stable and soluble peptidic folding precursor (Switch-Peptide) at physiological conditions, derived from a peptide/protein having a potential for self-assembling and fibrillogenesis.

Other objects and advantages will become apparent to those skilled in the art from a review of the ensuing detailed description, which proceeds with reference to the following illustrative drawings, and the attendant claims.

### BRIEF DESCRIPTION OF THE FIGURES

FIG. 1: Concept of the consecutive triggering of O, N-acyl migrations (AcM) in switch-peptides (Soff) for the onset (Son) of peptide folding and self-assembly in statu nascendi (ISN).

FIG. 2: Investigated switch-peptides and triggering systems (see FIG. 1). Aβ-sequences in square brackets. S=(Y₁₋₆)Ser/Thr (Ia, b); (Y₃)Cys (Ic); S₁/S₂=(Y₂/Y₁)Ser (IIa); (Y₃/Y₄)Ser (IIb); (Y₁/Y₃)Ser (IIc); Ψ=ΨPro [18]. Nomenclature depsipeptides see [19].

FIG. 3: A: CD of enzyme triggered (T₃) conformational transition of Ia. Inset: HPLC of time course, 1: S_{off}, 2: Sₒₙ, 3: ArgPro. B: HPLC of the sequential T₁/T₂-triggered acyl migration of IIa: 1: S_{1/2off}, 2: S_{1off/2on}; 3: intermediate 2 after cleavage of Y₂; 4: S_{1/2on}; inset: time course of hν cleavage (left) and CD (right) in H₂O/TFE (83/17). C: HPLC of the sequential T₁/T₃-triggered acyl migration of IIc: 1=S_{1/2off}, 2=S_{1on/2off}, 3: S_{1/2on}; inset: kinetics of acyl migrations for peptide IIb (T₃, T₄) and IIc (T₁, T₃).

FIG. 4: Amyloid β-derived switch-peptide 1 (SP1) with the switch-element at position 26 of A β (1-42).

FIG. 5: Solid phase synthesis of switch-peptide 1. (i) standard solid phase peptide synthesis according to the Fmoc/tBu strategy, (ii) 30 minutes coupling for Boc-Arg(di-Boc)-OH and Fmoc-Pro-OH; (iii) Fmoc-Gly-OH (3 eq), DIC (3 eq), DMAP (0.1 eq), DCM/DMF (4:1), 2 h; (iv) TFA/TIS/H₂O (94:2:2:2), 2×1 h.

FIG. 6: HPLC of crude and purified switch-peptide 1; m/z: 4764.

FIG. 7: Amyloid β-derived switch-peptide 2 (SP2) containing two switch-elements at position 26 and 37 of A β (1-42).

FIG. 8: Solid phase synthesis of switch-peptide 2. (i) Coupling with HATU; (ii) Coupling with PyBOP; (iii) Fmoc-Val-OH (3 eq), DIC (3 eq), DMAP (0.5 eq), DCM/DMF (4:1), 2×2 h; (iv) Fmoc-Gly-OH (3 eq), DIC (3 eq), DMAP (0.5 eq), DCM/DMF (4:1), 2×2 h; (v) TFA/TIS/EDT/H2O 94:2:2:2, 2×1 h.

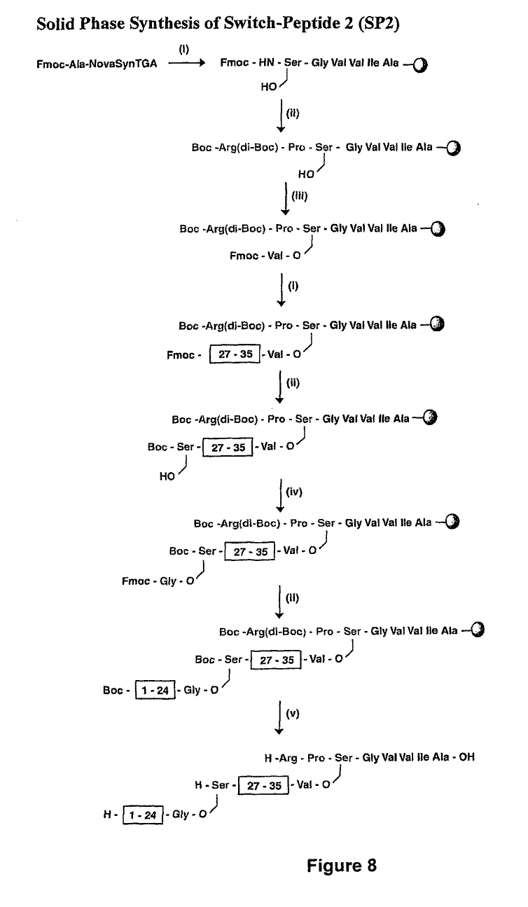

FIG. 9: a) HPLC chromatogram (C₈, 214 nm, 0→100% A, 30 min), b) MALDI-TOF mass spectrum of crude and purified switch-peptide 2.

FIG. 10: Host-guest switch-peptides as diagnostic tool for inhibitor screening, A: design of host-guest systems; B: quantitative assessment of inhibitory activity.

FIG. 11: Negatively stained electron microscopy performed on switch-peptide 2 after pH-induced (S1) and enzyme-triggered (S2) acyl migration and activation of S1 only.

FIG. 12: Overlay of the chromatograms obtained for the pH-induced acyl transfer at switch S1 in compound SP2. Peak 1 corresponds to switch-peptide 2 in the S_{off}-state, peak 2 represents SP2 after acyl migration at S1 restoring Aβ(1-36). Overlay of the HPLC chromatograms obtained for the DPPIV-triggered acyl transfer at S2 in switch-peptide 2. Peak 2 represents SP2 after acyl migration at switch S1, peak 3 corresponds to switch-peptide 2 after acyl migrations at S1 and S2.

FIG. 13: Conformational transition upon pH- and enzyme-triggered acyl migrations from a random coil (Soff) to a β-sheet (Son) of switch-peptide 2.

FIG. 14: HPLC chromatograms monitoring the chemical stability of switch-peptides at physiologic conditions (pH 7). O-acyl isopeptides are unstable at pH 7.4 undergoing spontaneous acyl migration. Switch-peptides are stable until acyl migration is induced upon enzyme triggering.

FIG. 15: Scheme of Applicant's screening system. Host-guest-switch-peptides (HGSP) as a molecular kit for the assessment of the inhibition of fibrillogenesis.

FIG. 16: Example for the inhibition of fibrillogenesis by peptidic inhibitors as monitored by CD. Peptide 5 (HGSP): Ac-SLSL-HQKLVFF-(H⁺)SEDV-SLG-NH₂; LS7: (LS)₃LG-NH₂; LS(Ψ)7: LSL-ΨPro-LSLG-NH₂.

### DETAILED DESCRIPTION OF THE INVENTION

As used herein, the following definitions are supplied in order to facilitate the understanding of the present invention.

The term “comprise” is generally used in the sense of include, that is to say permitting the presence of one or more features or components.

In the context of the present invention, “amyloid deposit” means any amyloid or amyloid-like deposit, including insoluble amyloid-beta-containing aggregates, such as plaques and fibrils.

As used herein, the terms “protein”, “polypeptide”, “polypeptidic”, “peptide” and “peptidic” or “peptidic chain” are used interchangeably herein to designate a series of amino acid residues connected to the other by peptide bonds between the alpha-amino and carboxy groups of adjacent residues.

“Amino acid residue” means any amino acid residue known to those skilled in the art. This encompasses naturally occurring amino acids (including for instance, using the three-letter code, Ala, Arg, Asn, Asp, Cys, Gln, Glu, Gly, His, Ile, Leu, Lys, Met, Phe, Pro, Ser, Thr, Trp, Tyr, Val), as well as rare and/or synthetic amino acids and derivatives thereof (including for instance Aad, Abu, Acp, Ahe, Aib, Apm, Dbu, Des, Dpm, Hyl, McLys, McVal, Nva, and the like.

Said amino acid residue or derivative thereof can be any isomer, especially any chiral isomer, e.g. the L- or D-isoform.

By amino acid derivative, we hereby mean any amino acid derivative as known in the art. For instance, amino acid derivatives include residues derivable from natural amino acids bearing additional side chains, e.g. alkyl side chains, and/or heteroatom substitutions.

“Fragments” refer to sequences sharing at least 40% amino acids in length with the respective sequence of the substrate active site. These sequences can be used as long as they exhibit the same properties as the native sequence from which they derive. Preferably these sequences share more than 70%, preferably more than 80%, in particular more than 90% amino acids in length with the respective sequence the substrate active site.

The present invention also includes variants of the substrate active site sequence. The term “variants” refer to polypeptides having amino acid sequences that differ to some extent from a native sequence polypeptide, that is amino acid sequences that vary from the native sequence by conservative amino acid substitutions, whereby one or more amino acids are substituted by another with same characteristics and conformational roles. The amino acid sequence variants possess substitutions, deletions, and/or insertions at certain positions within the amino acid sequence of the native amino acid sequence. Conservative amino acid substitutions are herein defined as exchanges within one of the following five groups:

I. Small aliphatic, nonpolar or slightly polar residues: Ala, Ser, Thr, Pro, Gly
II. Polar, positively charged residues: His, Arg, Lys
III. Polar, negatively charged residues: and their amides: Asp, Asn, Glu, Gln
IV. Large, aromatic residues: Phe, Tyr, Trp
V. Large, aliphatic, nonpolar residues: Met, Leu, Ile, Val, Cys.

It is an object of the present invention to provide a method for the manufacture of a stable and soluble peptidic folding precursor (Switch-Peptide) at physiological conditions (pH 7.0, Temperature 37° C.), derived from a peptide/protein having a potential for self-assembling and fibrillogenesis, wherein said method comprises the steps of:
generating at least one Switch Element (S-element) in the amino acid sequence of said peptide and,
subsequently reacting said modified peptide with an enzyme of the DPP IV family or enzyme having substantially the same specificity.

Enzymes that are used in the present invention are selected for their specificity toward the Switch Element binding site of formula:

H₂N-Axx-Pro-CO—

wherein Axx represent any proteinogenic or non proteininogenic amino acid residue. Preferably, Axx represents a basic or a hydrophobic amino acid residue. In particular, enzymes having specificity toward the Switch Element binding site are selected among enzymes that are listed in tables 3 or 4. Preferably, enzymes of the DPP IV family (dipeptidyl peptidase IV) are selected among the group comprising DPP IV, Esterase, Acylase, Phenacylase, Penicillin G amidase, D-aminopeptidase.

Preferably, the Switch Element is generated on a Serine, Cysteine, Threonine and/or any other conservative replacements by Ser, Cys, Thr in the native amino acid sequence of said peptide.

According to the present invention, the peptide having a potential for self-assembling and fibrillogenesis is able to adopt a beta-pleated sheet conformation and/or to form oligomers, fibrils and plaques. For example, peptides having a potential for self-assembling and fibrillogenesis are Amyloid beta, alpha synuclein, Huntington, Islet Amyloid protein or Prion proteins. The described procedures for transforming a native peptide/protein sequence to a switch-peptide as exemplified for amyloid β or analogs thereof can easily applied for this type of proteins.

Since an inherent problem with native peptides (in L-form) is degradation by natural proteases, the peptide precursor of the invention may be prepared to include D-forms and/or “retro-inverso isomers” of the peptide. In this case, retro-inverso isomers of short fragments and variants of the peptide of the invention are prepared.

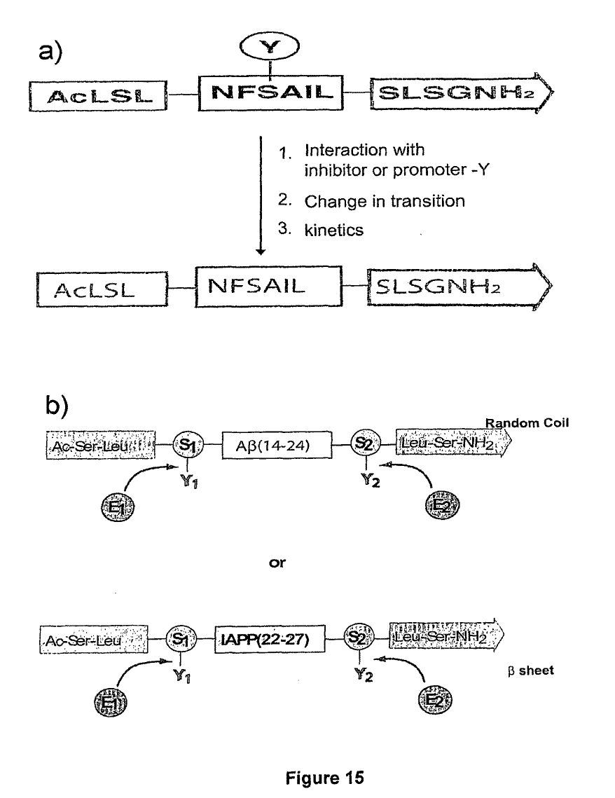

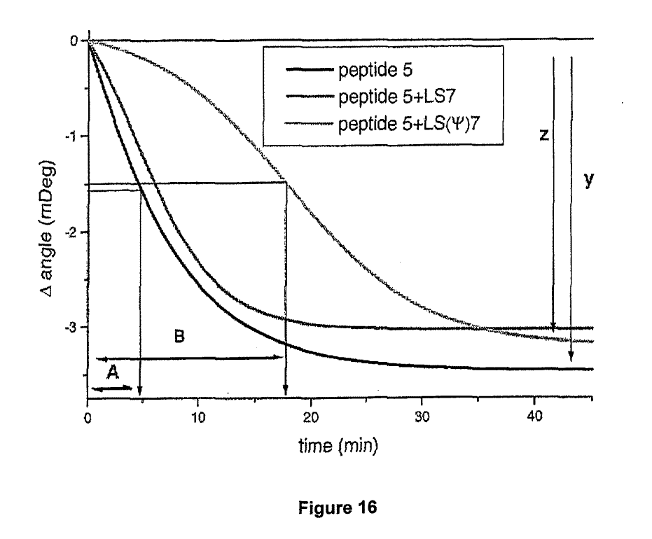

Also encompassed by the present invention is an Islet Amyloid polypeptide (IAPP) derived precursor molecule (Switch-Peptide) comprising the sequence N²²FGAIL²⁷ or analogs, variants or mutants thereof. Namely, two Switch elements are generated at the position N₂₂ and L₂₇ of the native peptide. The complete amino acid sequence of IAPP comprises 37 amino acids but the partial sequence N²²FGAIL²⁷ is considered as the core region for fibril formation and is inserted into a p-sheet and fibril promoting host-guest-switch-peptide (HGSP). These HGSPs are used as molecular kit systems for the screening of inhibitors as shown in FIGS. 15 and 16. It is the object of the present invention to provide a stable and soluble peptidic folding precursor (or Prodrug) derived from a peptide having a potential for self-assembling and fibrillogenesis, that it is obtained by the method of the invention. Peptides having a potential for self-assembling and fibrillogenesis are for example selected among the group comprising Amyloid beta, alpha synuclein, Huntingtin, Islet Amyloid protein or prion Proteins.

For the purposes of the present invention, a stable and soluble peptidic folding precursor or “prodrug” is an entity which represents an inactive form of an active peptide. In other words, the invention concerns a stable and soluble peptidic folding precursor (composition) which has the potential of producing a desired physiological effect on cells, but is initially inert (i.e. does not produce said effect), and only after undergoing some modifications becomes physiologically active and produces said physiological effect on cells. In particular, the stable and soluble peptidic folding precursor or “prodrug” of the present invention has a chemically or metabolically degradable group (Switch-Peptide), and becomes pharmaceutically active after biotransformation.

Biotransformation of the switch-peptide, according to the invention may be carried out under physiological conditions (in vitro and in vivo) and is a result of a reaction with an enzyme, or a body fluid such as gastric acid, blood etc., thus undergoing an enzymatic oxidation, reduction, hydrolysis etc. or a chemical hydrolysis to convert into the active compound by acyl migration reaction.

According to the present invention, pharmaceutically acceptable salts are produced from acidic inorganic or organic compounds, or alkaline inorganic or organic compounds. As used herein, the phrase “pharmaceutically acceptable salt” refers to a salt that retains the biological effectiveness of the free acids and bases of a specified compound and that is not biologically or otherwise undesirable.

Another object of the invention concerns a tool for the quantitative, controlled in vitro study of fibrillogenesis and its inhibition (inhibitor screening) of peptides/proteins involved in degenerative diseases comprising:
generating multi-switch peptide elements in a peptide sequence of a peptide able to form fibrillar aggregates,
switching on selectively, independently and orthogonaly said multi-switch elements one by one,
monitoring the resulting self-assembling, oligomerisation, aggregation and fibrillogenesis by analytical, spectroscopic or biophysical methods and/or in vitro tests and optionally assessing quantitatively inhibition (inhibitor screening) of these processes.

By multi-switch peptide elements it is intended at least two switch peptide elements. The switching on of said orthogonal multi-switch elements is carried out by reaction of an enzyme of the DPP IV family or enzyme having substantially the same specificity (see table 3 and/or 4).

For example, the monitoring is carried out by analytical (e.g. HPLC, Ultracentrifugation, turbidity), spectroscopic (e.g. CD, IR, NMR) or biophysical (e.g. transmission electron microscopy (TEM)) methods and/or in vitro tests (e.g. Thioflavin T, Congo red staining).

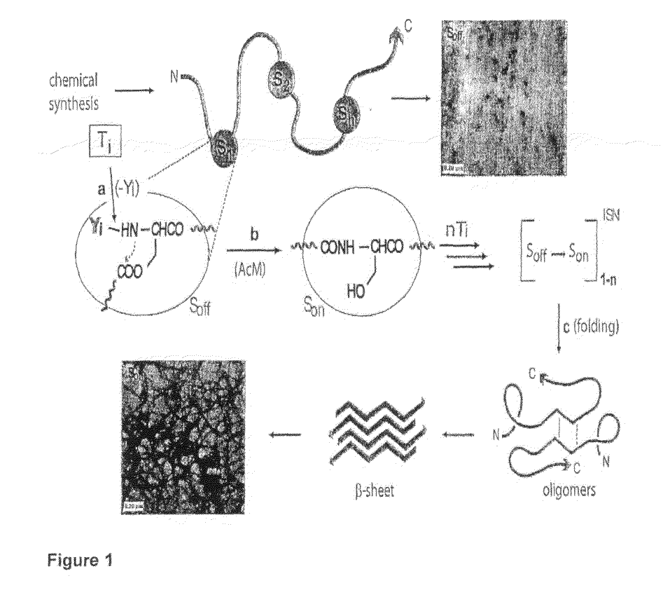

For potential applications in vitro and in vivo, Applicant now explores the sequential triggering of O→N-acyl migrations in amyloid β (Aβ) derived switch-peptides as a tool for studying onset and inhibition in polypeptide folding, self-assembly and aggregation. As shown in FIG. 1, N(Y)-protected O-acyl isopeptides (‘switch (S)-peptides’) serve as stable, self-contained precursor molecules, in which folding and self-assembly is blocked by the presence of the Ser, Thr or Cys-derived switch (S)-elements dissecting the regular peptide backbone by an ester and a flexible C—C bond (Soff).

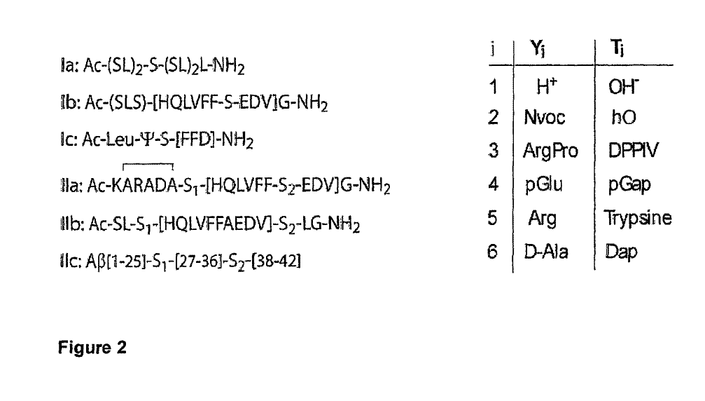

Here, Applicant focuses on the design and chemical synthesis of Aβ-derived S-peptides (FIG. 2) and investigates the specific cleavage of the N-protecting groups Y using chemical or enzymatic triggers T (step a, FIG. 1), the spontaneous intramolecular O→N acyl migration (step b) and the induction of folding events (step c) such as self-assembly, n-sheet and fibril formation in statu nascendi (ISN) of the molecule. For the selective removal of Y₁ by a trigger T, the use of exoproteases with ‘nonnative’ specificities such as pyroglutamate aminopeptidase (pGAP) and D-amino acid peptidase (Dap) or with unique cleavage sites such as dipeptidyl peptidase IV (DPPIV, specific for N-terminal Axx-Pro) are examined.

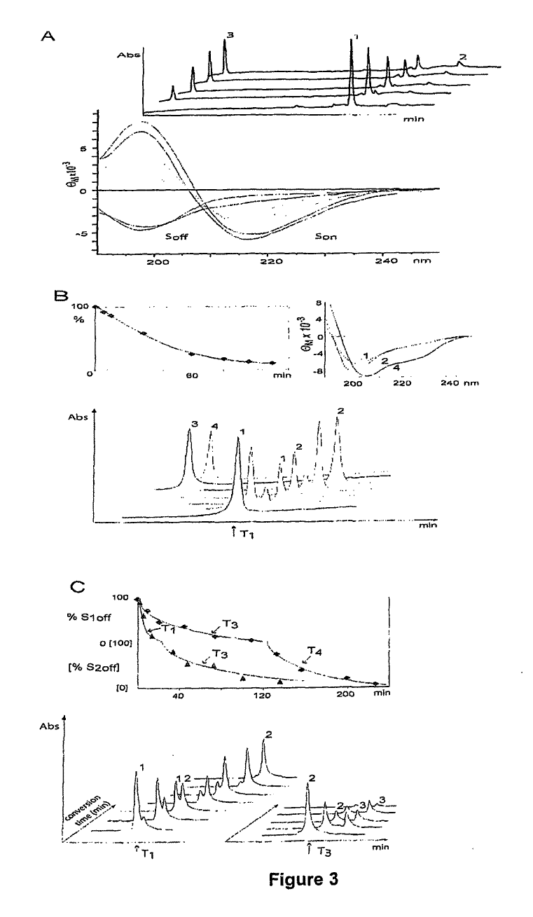

Solid-phase synthesis of I and II was achieved applying Fmoc/tBu-based chemistry, introducing the S-elements preferentially as N(Y)-protected depsidipeptides [9, 15]. Most notably, the presence of one (I) or two (II) S-elements results in highly soluble compounds facilitating HPLC purification and structural characterization. As shown by CD, the conformational decoupling of the S-spaced peptide blocks [6]) results in flexible random coil (rc) conformations (CD curves S_{off}, FIG. 3). Even after 24 h at physiologic pH, no changes in the HPLC and CD spectra are observed for the S_{off}-state of the S-peptides, pointing to high chemical and conformational stability.

In contrast, the controlled removal of Y in the individual S-elements provokes spontaneous intramolecular O, N-acyl migration, resulting in dramatic changes of the conformational and physical properties (Sₒₙ, state). For example, after adding enzyme DPPIV to S-peptides I, the evolution of the cleaved dipeptide Arg-Pro (FIG. 3A, HPLC peak 3), the gradual disappearance of the S_{off}-peak (1) as well as the onset of a new peak (2, Sₒₙ) reflect the overall time course for steps a and b, respectively (FIG. 1). As a general observation, the evolution and subsequent degradation of the Sₒₙ-peak points to fast aggregation originating from rc to β-sheet transitions (CD, FIG. 3A). As studied on series I, the time course for the process S_{off}→Sₒₙ strongly depends on the triggering system (minutes up to several hours in going from trigger systems 1 to 6), whereas the intramolecular O, N-acyl transfer reaction proceeds generally fast at physiologic pH (Thr≦Ser<<Cys).

The consecutive ‘switching on’ of S-elements according to FIG. 1 provides an experimental tool for evaluating the impact of individual peptide segments upon folding and self-assembly. For example, the pH-induced acyl migration at S₂ in IIa (HPLC, FIG. 3B) does not result in a significant effect upon the CD spectra (predominant rc structure), whereas the switching on of the helix-nucleating system [16] by photolytic cleavage at S₁ and subsequent acyl migration induces helical conformation (inset (right) FIG. 3B). Notably, photolytic cleavage at acidic pH allows to independently monitor step a (peak 3, S_{off}, FIG. 1B; inset (left): time course) and step b (Sₒₙ, peak 4), opening interesting applications for the use of orthogonal switch arrays in organic and aqueous solvents. Selective switching on of the N- and C-terminal sequence in IIb is achieved upon consecutive addition of triggers T₃ and T₄, respectively (inset FIG. 3C, time course). Again, the sequential order of triggering acyl migrations proves to be essential: setting off the N-terminal Ac-SerLeu by adding T₃ does not affect the overall properties of the peptide (rc conformation, solubility), whereas a conformational transition of type rc→β-sheet, followed by aggregation, is induced upon ligating the C-terminal SerLeuGlyNH₂ (applying T₄), thus providing interesting clues for the onset of β-sheets [6].

Finally, the consecutive switching on of peptide segments is exemplified for [Ser³⁷] Aβ(1-42) containing a chemical (S₁) and enzymatic cleaving (S₂) site (IIc). Here, the pH-induced acyl migration at S₁ proceeds fast (t_{1/2}=5 min, inset, FIG. 1C) restoring native Aβ (1-36) (HPLC, peak 2, FIG. 3C). Interestingly, by the subsequent enzymatic switching on (T₃, inset FIG. 3C) of the C-terminal segment (37-42), the characteristic phenomena observed for native Aβ (1-42) [1-5], i.e. β-sheet and fibril formation are initiated, accompanied by self-association and aggregation (disappearance of Sₒₙ peak 3, FIG. 3C). Though these observations will be subject of detailed conformational analyses [17], our preliminary CD and TEM studies point to the central impact of the hydrophobic C-terminus of Aβ (1-42) upon self-association and aggregation, setting the stage for a rational design of specific inhibitors [18].

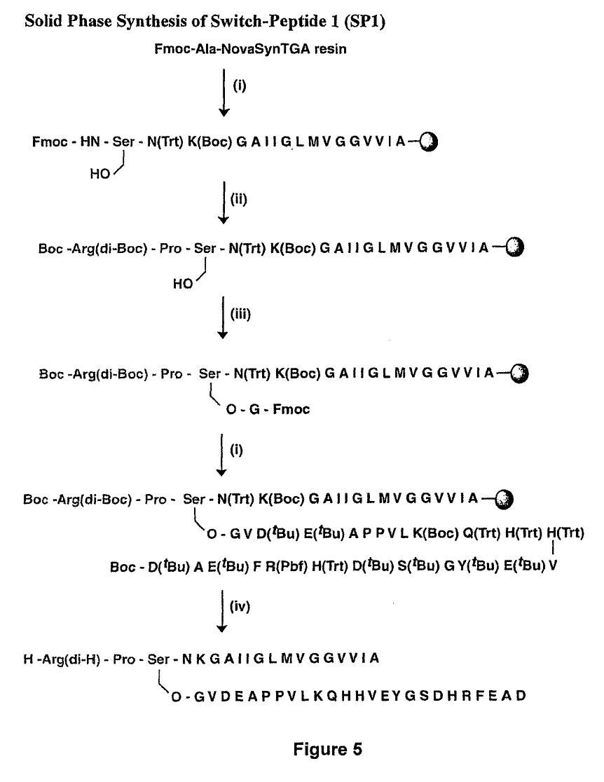

Yet another object of the invention is to provide for a Host-guest switch peptide molecular system for the screening of inhibitors of peptides involved in degenerative diseases. This in vitro system for the screening of the inhibitory effect of β-breaker molecules acting on peptides able to form fibrillar aggregates and involved in degenerative diseases comprising:
1) generating a host-guest-switch-peptide containing one or two switch-elements (Soff-state)
and comprized of a fibril nucleation sequence (guest) and a beta-sheet promoting host according to FIG. 15.
2) Adding a potential inhibitor (or a library of potential inhibitors) in a buffer solution for example at pH<5 (pH-triggered switch-element) or at physiologic conditions (enzyme-triggered S-element).
3) Triggering the acyl migration (-Y in FIG. 5) according to the general concept of S-peptides described herein.
4) Monitoring the resulting self-assembling, oligomerisation, aggregation and fibrillogenesis by analytical, spectroscopic or biophysical methods and/or in vitro tests.

In summary, the in vitro system for the screening of the inhibitory effect of β-breaker or fibril disrupting molecules acting on peptides able to form fibrillar aggregates and involved in degenerative diseases comprises:
a) a Host-guest-switch-peptide (HGSP) containing one or two switch-elements (Soff-state) and,
b) a potential of β-sheet inhibitor and/or fibril disrupting molecule or libraries thereof and,
monitoring the resulting self-assembling, oligomerisation, aggregation and fibrillogenesis after chemical and/or enzymatic triggering of acyl migration (Son) by analytical, spectroscopic or biophysical methods and/or in vitro tests.

Examples of peptides able to form fibrillar aggregates and involved in degenerative diseases are for example: Amyloid beta, alpha synuclein, Huntingtin, Islet Amyloid protein or prion Proteins and are transformed to host-guest switch-peptides according to the invention.

The present invention provides a novel strategy, using ‘host-guest switch-peptides’ to monitor fibril formation and thereby screening the inhibitory effect of fibril disrupting molecules. Typically, HQKLVFFAED and NFGAIL, the partial sequences of Aβ (Aβ 14-24) and amylin (amylin 17-22) peptide, respectively, are known to play a key role in the nucleation of their aggregation and are used as guest sequences (FIG. 15). However, these small peptide fragments do not show n-sheet formation in aqueous solution as monitored by CD. On the other hand, oligopeptides of alternative hydrophobic and hydrophilic amino acid, for example (Leu-Ser)ₙ, exhibit high potential for β-sheet formation. To mimic the aggregation phenomena of native AD and amylin peptide, a host-guest peptide is designed, inserting HQKLVFFAED and NFGAIL in Leu-Ser oligomers as a host. This host-guest peptide indeed forms β-sheet structure in aqueous solution leading to amyloid-like aggregation. To properly initiate the conformational transition from a flexible random coil conformation, a serine switch is introduced in the middle (FIG. 15a) (alternatively, at the C- and/or N-terminal end of the guest peptide, FIG. 15b), replacing Ala²¹ of Aβ and Gly¹⁹ of amylin, thus providing host-guest switch-peptides (HGSP). By triggering the acyl migration upon changing the pH (alternatively, by enzymatic triggering, depending on Y), these peptides are designed to convert, from a non functional (S_{off}) to a functional (Sₒₙ) state, as schematically shown in FIG. 15. The rate of conversion can be controlled by changing pH, temperature and solvent system. Incubating the β-breaker (or fibril disrupting) molecules with these HGSP, prior to the triggering of the switch, should influence the kinetics of the conformational transition, hence the aggregation of the HGSP. By comparing the kinetics of aggregation of HGSP in the presence or absence of the β-breaker molecules, one can determine the efficiency of the β-breaker compound (FIG. 15) inhibitor screening system.

For example, after HGSP incubation with inhibitor compounds, the switch is triggered chemically or enzymatically and the process of β-sheet or fibril formation is monitored by CD or any alternative biophysical method for detecting self-association processes. The change in the kinetics of the process relative to the inhibitor free reference system gives the information about the efficiency of the inhibitor.

This type of screening system seems ideal for this purpose, due to the following reasons: (a) One can be sure about the absence of polymerized forms at t=0, as at the time of triggering the switch, the HGSP is in S_{off} state, which is essentially in a random coil conformation. (b) It is possible to control the kinetics of the aggregation, either by changing the experimental conditions or by modifying the host-guest sequence. (c) As the HGSPs are smaller than the corresponding native sequences, these compounds are readily prepared in contrast to the use of full length Aβ or amylin peptide. (d) CD spectrometry proves to be the method of choice for getting the information about inhibition at early stages of self-association. This is an important point, because for investigating the β-breaking potential of β-breakers, one should focus on monitoring the indicated conformational transitions. Using HGSP allows following the early stage of aggregation, originating from conformational changes. In summary, the designed system of HGSP appears most versatile for a preliminary screening of β-breaker compounds.

Finally, also encompassed are β-breaker inhibitors or fibril disrupting molecules of a peptide involved in degenerative diseases, obtainable from the in vitro system according to the invention.

The advantages of Enzyme-Triggered Acyl Migrations ('Switch-peptides' as tool) compared to the state-of-the-art (pH-triggered acyl migrations, e.g. Y. Kiso et al, L. Carpino et al.) are numerous.

The inventive method according to the invention, facilitates solubility and synthesis (multiple S); Y=group (e.g. Arg, His, Lys); strongly solubilising.

It is now possible to store peptidic folding precursors at neutral conditions (physiologic) and stability is increase with pH (peptidic folding precursors more stable at pH 7) compared to Y. Kiso et al. (pH 4, protonated form)

Applicant's screening system also provides a molecular kit system at physiologic pH for use in vitro and in vivo. The Addition of enzyme at physiologic pH starts onset of fibril formation under controlled conditions (no change of pH necessary, which is critical to standardize).

Inhibitor can be added to switch-peptide first (e.g. S_{off}-state of switch-Aβ) and equilibrated at physiologic conditions. Fibrillization is started by adding enzyme and the determination of the inhibition factor is possible.

The method may be used for sequential triggering by using specific enzymes for corresponding substrate Y (S₁, S₂ . . . ), see Table 3 and 4 of triggering systems.

Applicant's in vitro system may contain multiple S-elements (triggered by DPPIV, GAP, Acylase, Phenacylase, D-aminopeptidase) for the screening of sequence-specific inhibitors and for studying the mechanism of folding and self-assembly.

In summary, the invention presents a surprising and novel concept for the controlled, sequential onset of peptide assembly in vitro. In particular, the enzymatic triggering (see Table 3 and 4) of O, N-acyl migrations allows for novel applications in prodrug design and biosensor technology. In further exploring the immense potential of peptide and protein synthesis, switch-peptides may become a general tool for the study of early steps in polypeptide self-assembly and inhibition as key process in degenerative diseases.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended claims, and all changes which come within the meaning and range of equivalency are intended to be embraced therein.

Various references are cited throughout this Specification, each of which is incorporated herein by reference in its entirety.

The foregoing description will be more fully understood with reference to the following Examples. Such Examples, are, however, exemplary of methods of practising the present invention and are not intended to limit the scope of the invention.

### EXAMPLES

### General

Amino acid derivatives were purchased from Merck-Novabiochem or Alexis (both Läufelfingen, Switzerland) and Bachern Fine Chemicals (Bubendorf, Switzerland). Solvents and reagents were purchased from Fluka (Buchs, Switzerland), Sigma-Aldrich Chemie GmbH (Steinheim, Germany) or Acros (Geel, Belgium) and used without further purification. DMF for peptide synthesis was purchased from SdS (Peypin, France) and degassed with nitrogen before use. Acetonitrile for HPLC was obtained from Biosolve BV (Valkenswaard, Netherlands). Water used for HPLC was of Milli-Q quality, collected after passing through a Millipore purification system (Volketswil, Switzerland). Trifluoroacetic acid for HPLC was purchased from Baker AG (Basel, Switzerland).

MALDI-TOF mass spectra were recorded on a matrix-assisted laser desorption/ionization time of flight mass spectrometer Axima-CFR Shimadzu (Duisburg, Germany) using a-cyano-4-hydroxy cinnamic acid as a matrix.

ESI-MS was performed on a Finnigan MAT SSQ 710C electrospray spectrometer equipped with an IBM PS/2 95 XP 486 (software Technivent Vector II) in positive ionization mode, at 200° C. capillary temperature and a potential of 4.5 kV.

Analytical reversed-phase HPLC spectra were recorded on a Waters system (Waters Corporation, Milford, Mass., USA) consisting of two Waters 600 pumps, a Waters 600 System Controller, a Waters 486 Tunable Absorbance Detector and a printer Waters 746, using columns packed with Vydac Nucleosil 218TP54 C₁₈ particles (250×4.6 mm), or 208TP54 C₈ particles (250×4.6 mm). Flow rates of 1 mL/min were used and the UV absorbance was monitored at 214 nm. All gradients were linear in eluent A (0.09% TFA in 90% aqueous acetonitrile) and eluent B (0.09% TFA in water).

Preparative HPLC purifications were performed on a Waters Delta Prep 3000 System, with a Waters 600E System Controller and a Waters 484 Absorbance Detector, with Vydac Nucleosil 218TP152050R C₁₈ particles (5×25 cm). Flow rates of 80 mL/min were used and the UV absorbance was monitored at 214 nm. All gradients were linear in eluent A (0.09% TFA in 90% aqueous acetonitrile) and eluent B (0.09% TFA in water).

Semi-Preparative HPLC purifications were performed on a Waters Delta Prep 3000 System, with a Waters 600E System Controller and a Waters 484 Absorbance Detector, with Vydac Nucleosil 218TP1022 C₁₈ particles (22 mm), or 208TP1022 C₈ particles (2.2×25 cm). Flow rates of 18 mL/min were used and the UV absorbance was monitored at 214 nm. All gradients were linear in eluent A (0.09% TFA in 90% aqueous acetonitrile) and eluent B (0.09% TFA in water). For column chromatography, silica gel SiO₂ Merck 60 (0.040-0.063 mm, 230-400 Mesh) was used.

CD spectra were recorded on JASCO J-810 spectropolarimeter. A supply of high-purity nitrogen is essential to displace oxygen, in order to avoid degradation of the mirrors by ozone generated by the high power xenon source as well as to reduce absorbance from oxygen bands below 200 nm. A 1 mm cuvette (Hellma, QS, strain-free suprasil) was used for all measurements. The cell was cleaned prior to each use, with TFA or conc. chromic acid, water and methanol and dried using a jet of nitrogen gas. Spectra were recorded from 190 nm to 250 nm using Time course or Interval scan measurements. Scanning mode was continuous with a speed of 100 nm/min, a 0.25 s response, a bandwidth of 1 nm, an accumulation of 1 and a data pitch of 0.2 nm.

### 1) Synthesis of Depsi-Dipeptide

### Fmoc-Tyr(^{t}Bu)-(Boe)Thr-OBzl

N-(tert-Butoxycarbonyl)-L-threonine benzyl ester 1: To a solution of L-Threonine benzyl ester oxalate (2 g, 6.682 mmol, 1 eq) in 66 mL of dry DMF, 1.65 mL (13.365 mmol-2 eq) of DIPEA and 1.31 g (6.014 mmol, 0.9 eq) of di-tert-butyl dicarbonate were added at 0° C. The reaction was stirred overnight at room temperature. After evaporation of DMF, the crude was diluted in AcOEt, and successively washed with a solution of citric acid (6%), a saturated solution of NaHCO₃ and brine. The organic solution was dried over MgSO₄ and the solvent evaporated. The crude product was purified by silica gel chromatography (AcOEt/Hexane 4:6) to yield 1.98 g (quantitative) colorless oil.

HPLC(C₁₈, 214 nm, 0→100% A, 30 min): Rₜ=22.5 min.

ESI-MS: m/z=309.83 [M+H]⁺, 619.32 [2M+H]⁺.

Fmoc-Tyr(^{t}Bu)-(Boc)Thr-OBzl 2: To a solution of Fmoc-Tyr(^{t}Bu)-OH (2.94 g, 6.407 mmol, 1 eq) in 30 mL of dry THF, 1.89 g (6.407 mmol, 1 eq) of MSNT were added at 0° C. After 30 min at room temperature, 1.98 g (6.407 mmol, 1 eq) of compound 1 diluted in 30 mL of THF and 254 μL of MeIm (3.203 mmol, 0.5 eq) were added dropwise at 0° C. The cooling bath was removed and the reaction stirred overnight at room temperature. After evaporation of THF, the crude was diluted in DCM, and successively washed with a solution of citric acid (6%) and brine. The organic solution was dried over MgSO₄ and the solvent evaporated. The crude product was purified by silica gel chromatography (AcOEt/Hexane 3:7) to yield a white powder (2.5 g, 53%).

HPLC(C₁₈, 214 nm, 50→100% A, 30 min): Rₜ=29.7 min.

ESI-MS: m/z=751.33 [M+H]⁺.

### 2) Solid-Phase Peptide Synthesis: General Procedure

The syntheses were carried out manually on a pre-loaded Fmoc-Ala-NovaSyn TGA (Tentagel) resin (composite of polyethylene oxide grafted onto a low cross-linked polystyrene gel-type matrix) following the Fmoc/tBu strategy [20].

The Fmoc-amino acid side-chain protections were selected as follows: tBu (Asp, Glu, Ser, Thr, Tyr), Boc (Lys, Orn, Arg), Pbf (Arg), Trt (Asn, Gln, His). Generally, the peptide chains were assembled by sequential coupling of activated Na-Fmoc amino acid (2.0 or 4 eq.) in DMF (1.5-2.0 mL) in the presence of (benzotriazol-1-yloxy)-tris(pyrrolidino)phosphonium hexafluorophosphate (PyBOP, 2 eq.), N[(dimethylamino)-1H-1,2,3-triazol [4,5-b]pyridin-1-yl-methylene]-N-methylmethan aminium hexafluorophosphate N-oxide (HATU, 2 or 4 eq.) or 1,3-diisopropoylcarbodiimide (DIPCDI, 3 eq.) with a reaction time of 1 h at room temperature. The peptide-resins were washed with DMF (5 times) and the completeness of each coupling was verified by the Kaiser test or test cleavages of the peptide from the resin. Nα-deprotection was carried out by treatment with piperidine (20% v/v in DMF), 2% 1,8-diazabicyclo[5.4.0]-undec-7-ene (DBU, v/v in DMF) or a mixture of 2% DBU and 5% piperidine in DMF (2 mL, 1 min (1×) and 20 min (1×)), followed by washing with DMF (1.5 mL, 10×) and methylene chloride (1.5 mL, 5×). If necessary, the coupling and deprotection cycles were repeated. After the peptide resins were washed with methanol (1.5 mL, 5×) and dried for 2 h in vacuo, the peptides were cleaved from the resin with TFA in the presence of triisopropylsilane (TIS), ethanedithiol (EDT) and distilled water (94:2:2:2) for 60 min (2×) at room temperature, concentrated in vacuo, and precipitated with diethyl ether (4-8 mL) at 0° C. followed by centrifugation at 3000 rpm for 5 min (3×). The resultant peptides were dissolved in H₂O and the crude products purified by preparative reversed-phase HPLC with 0.09% aqueous TFA-CH₃CN system as eluant, frozen at −78° C. and lyophilized at least 12 h. Purified peptides were stored at −20° C. until use.

### 3) Switch-Peptide 1 (SP1):

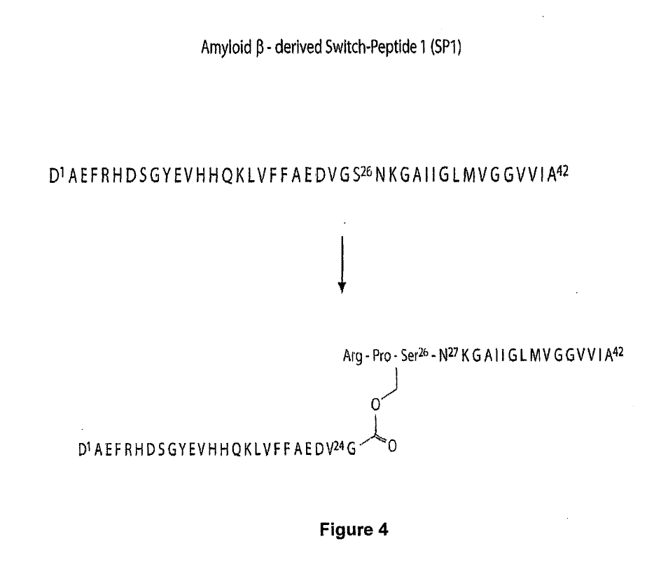

### Amyloid β (1-25)-S₁-(27-42) [S₁=(ArgPro)Ser] (see FIGS. 4/5; Table 1)

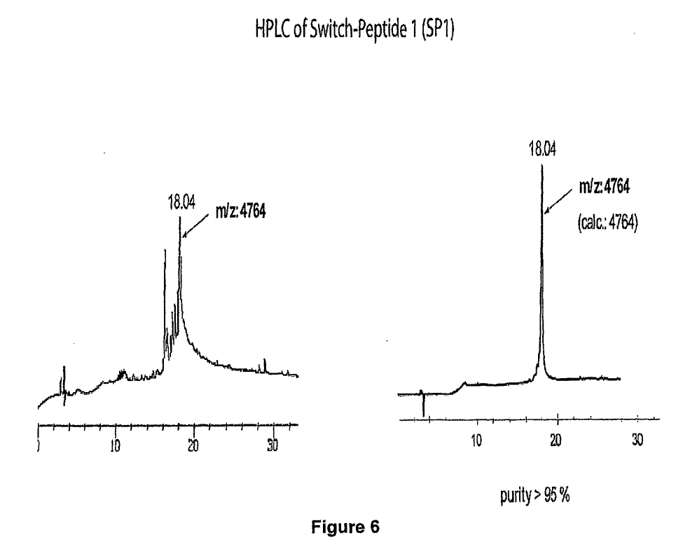

Synthesis was performed manually on 220 mg (0.05 mmol) of a pre-loaded Fmoc-Ala-NovaSyn TGA resin (loading 0.23 mmol/g). The Fmoc protecting group was removed with 20% piperidine in DMF (10 min, 2×) and the peptide-resin washed with DMF (5×) and CH₂Cl₂ (3×). The amino acid Fmoc-Ile-OH (36 mg, 0.1 mmol) was coupled with 2 eq PyBOP for 1.5 h. After washing with DMF (1.5 mL, 5×), the Fmoc group was removed with 20% piperidine in DMF and Fmoc-Val was coupled (34 mg, 0.1 mmol) in the presence of 2 eq. (0.1 mmol) PyBOP for 45 min. The sequential Fmoc-protected amino acids (0.1 mmol) were coupled in the presence of HATU (2 eq., 0.1 mmol) for 45, 60 or 90 min in DMF (1.5 mL) after removal of each Fmoc group by 2% DBU in DMF up to residue 28 (Asn). Fmoc-Ser-OH (residue 27) (33 mg, 0.1 mmol) was coupled in the presence of PyBOP (2 eq.) for 1 h in DMF (1.5 mL). The sequential Fmoc groups were removed with a mixture of 2% DBU and 5% piperidine in 2 mL DMF [1 min (1×) and 20 min (1×)] and Fmoc-Pro-OH (36 mg, 0.1 mmol) and Boc-Arg(di-Boc) (48 mg, 0.1 mmol) coupled successively with 2 eq. PyBOP for 30 min. Coupling with Fmoc-Gly-OH (45 mg, 0.15 mmol) was performed using the DIPCDI (3 eq., 0.15)—DMAP (0.1 eq.) method in DMF/CH₂Cl₂ (2:3, v/v) for 2 h (2×). Subsequent amino acid residues (4 eq., 0.2 mmol) were coupled with HATU (4 eq., 0.2 mmol, 1 h) after the removal of each Fmoc group using 2% DBU and 5% piperidine in DMF [2 mL, 1 min (1×) and 20 min (1×)] or 20% piperidine in DMF. The Nα-Fmoc deprotection of the last 7 N-terminal amino acid residues was carried out by treatment with piperidine (20% v/v) in DMF. Resulting protected peptide-resin (352 mg) was treated with TFA (4.7 mL):TIS (100 μL):EDT (100 μL):H₂O (100 μL) for 60 min (2×), concentrated in vacuo, washed with diethylether, centrifuged, dissolved in water and lyophilized to give the crude O-acyl isopeptide (SP1) (80 mg, 34%). The crude peptide was dissolved in water, applied to preparative HPLC, and eluted using 0.09% aqueous TFA-CH₃CN(C₈, 214 nm, 20-40%, 30 min). The desired fractions were collected and lyophilized to afford SP1 as a white amorphous powder. Yield: 31.3 mg (13%); MALDI-TOF MS: M_{calc}: 4763.2; M+H_{found}: 4764; HPLC analysis at 214 nm: purity was >95% (FIG. 6).

TABLE 1
Detailed protocol of the solid phase peptide synthesis of switch-peptide 1.

| | Quantity | | | Fmoc |
|---|---|---|---|---|
| Residue | (mg) | Coupling conditions | Test | deprotection |
| Fmoc-Ile-OH | 36 | PyBOP, 2eq, 90 min | Kaiser (-) | 20% piperidine |
| Fmoc-Val-OH | 34 | PyBOP, 2eq, 45 min | Kaiser (-) | 2% DBU |
| Fmoc-Val-OH | 34 | HATU, 2eq, 45 min | Kaiser (-) | 2% DBU |
| Fmoc-Gly-OH | 30 | HATU, 2eq, 45 min | Kaiser (-) | 2% DBU |
| Fmoc-Gly-OH | 30 | HATU, 2eq, 45 min | Kaiser (-) | 2% DBU |
| | | | Cleavage | |
| | | | Rₜ = 23.6 min | |
| | | | [M + H]⁺ = 737 | |
| Fmoc-Val-OH | 34 | HATU, 2eq, 45 min | Kaiser (-) | 2% DBU |
| Fmoc-Met-OH | 38 | HATU, 2eq, 90 min | Kaiser (-) | 2% DBU |
| Fmoc-Leu-OH | 36 | HATU, 2eq, 45 min | Kaiser (-) | 2% DBU |
| Fmoc-Gly-OH | 30 | HATU, 2eq, 90 min | Kaiser (-) | 2% DBU |
| Fmoc-Ile-OH | 36 | HATU, 2eq, 60 min | Cleavage | 2% DBU |
| | | | Rₜ = 23.2 min | |
| | | | [M + H]⁺ = 1250 | |
| Fmoc-Ile-OH | 36 | HATU, 2eq, 45 min | Kaiser (-) | 2% DBU |
| Fmoc-Ala-OH•H₂O | 32 | HATU, 2eq, 60 min | Kaiser (-) | 2% DBU |
| Fmoc-Gly-OH | 30 | HATU, 2eq, 60 min | Kaiser (-) | 2% DBU |
| Fmoc-Lys(Boc)-OH | 48 | HATU, 2eq, 60 min | Cleavage | 2% DBU |
| | | | Rₜ = 17.7 min | |
| | | | [M + 2H]²⁺ = 810 | |
| Fmoc-Asn(Trt)-OH | 61 | HATU, 2eq, 60 min | Kaiser (-) | 2% DBU |
| Fmoc-Ser(OH)—OH | 33 | PyBOP, 2eq, 60 min | Kaiser (-) | 2% DBU |
| | | | | 5% piperidine |
| Fmoc-Pro-OH•H₂O | 36 | PyBOP, 2eq, 30 min | Kaiser (-) | 2% DBU |
| | | | | 5% piperidine |
| Boc-Arg(di-Boc)-OH | 48 | PyBOP, 2eq, 30 min | Cleavage | — |
| | | | Rₜ = 17.7 min | |
| | | | [M + 2H]²⁺ = 926 | |
| | | | [M + 3H]³⁺ = 618 | |
| Fmoc-Gly-OH | 45 | CDI, 3eq, 2*120 min | Cleavage | 2% DBU |
| | | | [M + 2H]²⁺ = 1065 | 5% piperidine |
| | | | [M + 3H]³⁺ = 711 | |
| Fmoc-Val-OH | 34 | HATU, 2eq, 60 min | Kaiser (-) | 2% DBU |
| Fmoc-Asp(^{t}Bu)-OH | 83 | HATU, 4eq, 60 min | Kaiser (-) | 2% DBU |
| | | | | 5% piperidine |
| Fmoc-Glu(^{t}Bu)-OH | 90 | HATU, 4eq, 60 min | Kaiser (-) | 2% DBU |
| | | | | 5% piperidine |
| Fmoc-Ala-OH•H₂O | 64 | HATU, 4eq, 60 min | Kaiser (-) | 2% DBU |
| | | | | 5% piperidine |
| Fmoc-Phe-OH | 79 | HATU, 4eq, 60 min | Cleavage | 2% DBU |
| | | | [M + 3H]³⁺ = 898 | 5% piperidine |
| Fmoc-Phe-OH | 79 | HATU, 4eq, 60 min | Kaiser (-) | 2% DBU |
| | | | | 5% piperidine |
| Fmoc-Val-OH | 68 | HATU, 4eq, 60 min | Kaiser (-) | 2% DBU |
| | | | | 5% piperidine |
| Fmoc-Leu-OH | 72 | HATU, 4eq, 2*60 min | Cleavage | 2% DBU |
| | | | [M + 3H]³⁺ = 1017 | 5% piperidine |
| Fmoc-Lys(Boc)-OH | 96 | HATU, 4eq, 2*60 min | Kaiser (-) | 2% DBU |
| | | | | 5% piperidine |
| Fmoc-Gln(Trt)-OH | 124 | HATU, 4eq, 2*60 min | Kaiser (-) | 2% DBU |
| | | | | 5% piperidine |
| Fmoc-His(Trt)-OH | 125 | HATU, 4eq, 2*60 min | Cleavage | 2% DBU |
| | | acetylation | Rₜ = min | 5% piperidine |
| | | | [M + 4H]⁴⁺ = 862 | |
| | | | [M + 5H]⁵⁺ = 690 | |
| Fmoc-His(Trt)-OH | 125 | HATU, 4eq, 60 min | Kaiser (-) | 2% DBU |
| | | | | 5% piperidine |
| Fmoc-Val-OH | 68 | HATU, 4eq, 60 min | Kaiser (-) | 2% DBU |
| | | | | 5% piperidine |
| Fmoc-Glu(^{t}Bu)-OH | 90 | HATU, 4eq, 60 min | Kaiser (-) | 2% DBU |
| | | | | 5% piperidine |
| Fmoc-Tyr(^{t}Bu)-OH | 93 | HATU, 4eq, 60 min | Kaiser (-) | 2% DBU |
| | | | | 5% piperidine |
| Fmoc-Gly-OH | 60 | HATU, 4eq, 60 min | Kaiser (-) | 2% DBU |
| | | | | 5% piperidine |
| Fmoc-Ser(^{t}Bu)-OH | 78 | HATU, 4eq, 60 min | Kaiser (-) | 2% DBU |
| | | | | 5% piperidine |
| Fmoc-Asp(^{t}Bu)-OH | 83 | HATU, 4eq, 60 min | Kaiser (-) | 20% piperidine |
| Fmoc-His(Trt)-OH | 125 | HATU, 4eq, 60 min | Kaiser (-) | 20% piperidine |
| Fmoc-Arg(Pbf)-OH | 131 | HATU, 4eq, 60 min | Kaiser (-) | 20% piperidine |
| Fmoc-Phe-OH | 79 | HATU, 4eq, 60 min | Kaiser (-) | 20% piperidine |
| Fmoc-Glu(^{t}Bu)-OH | 90 | HATU, 4eq, 60 min | Kaiser (-) | 20% piperidine |
| Fmoc-Ala-OH•H₂O | 64 | HATU, 4eq, 60 min | Kaiser (-) | 20% piperidine |
| Boc-Asp(^{t}Bu)-OH | 60 | HATU, 4eq, 60 min | — | — |

Solid Phase Synthesis Protocol SP1
Fmoc-Ala-NovaSyn TGA resin 220 mg, loading 0.23 mmol/g

### 4) Switch-Peptide 2 (SP2):

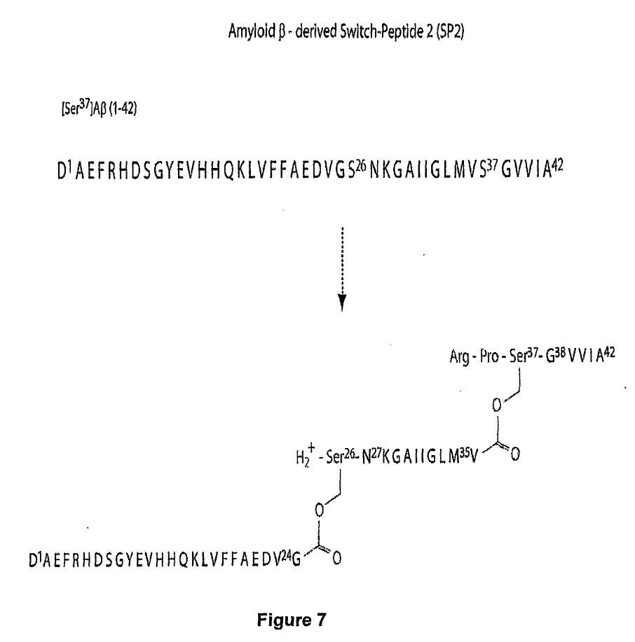

### Amyloid β (1-25)S₁-(27-36)S₂-(38-42) [S₁=(H₂⁺)Ser; S₂=(ArgPro)Ser] (see FIGS. 7/8; Table 2)

SP2 was synthesized in the same manner as described in the synthesis of SP1 on Fmoc-Ala-NovaSyn TGA resin (300 mg, 0.069 mmol; loading 0.23 mmol/g). Boc-Ser-OH (2 eq., 28 mg, 0.14 mmol), was coupled using 2 eq. PyBOP for 1 h. Coupling with Fmoc-Gly-OH (3 eq., 62 mg, 0.21 mmol) was performed using DIPCDI (3 eq.) and DMAP (0.1 eq.) in DMF/CH₂Cl₂ (2:3, v/v) for 2 h (2×).

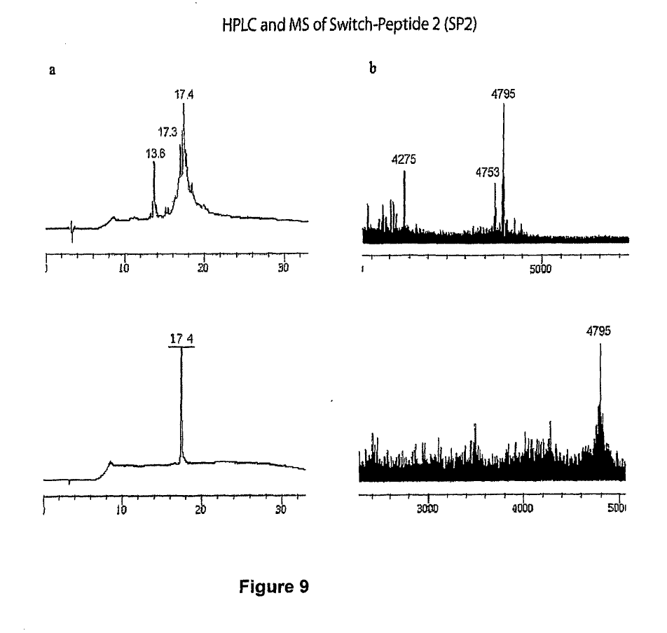

Resulting protected peptide-resin (445 mg) was treated with TFA (4.7 mL):TIS (100 μL):EDT (100 μL):H₂O (100 μL) for 60 min (2×), concentrated in vacuo, washed with diethylether, centrifuged, dissolved in water and lyophilized to give the crude O-acyl isopeptide (SP1) (100 mg, 30%). The crude peptide was dissolved in water, applied to preparative HPLC, and eluted using 0.09% aqueous TFA-CH₃CN(C₈, 214 nm, 20-40%, 30 min). The desired fractions were collected and lyophilized to afford SP2 as a white amorphous powder. Yield: 35.8 mg (10.8%); MALDI-TOF MS: M_{calc}: 4794.1; M+H_{found}: 4795; HPLC analysis at 214 nm: purity was >96% (FIG. 9).

TABLE 2
Detailed protocol of the solid phase peptide synthesis of switch-peptide 2.

| | Quantity | | | Fmoc |
|---|---|---|---|---|
| Residue | (mg) | Coupling conditions | Cleavage test | deprotection |
| Fmoc-Ile-OH | 49 | PyBOP, 2eq, 60 min | — | 20% piperidine |
| Fmoc-Val-OH | 47 | PyBOP, 2eq, 60 min | — | 2% DBU |
| Fmoc-Val-OH | 47 | HATU, 2eq, 60 min | — | 2% DBU |
| Fmoc-Gly-OH | 41 | HATU, 2eq, 60 min | — | 2% DBU |
| Fmoc-Ser(OH)—OH | 45 | PyBOP, 2eq, 60 min | — | 2% DBU |
| Fmoc-Pro-OH•H₂O | 49 | PyBOP, 2eq, 60 min | | 2% DBU |
| Boc-Arg(di-Boc)-OH | 66 | PyBOP, 2eq, 60 min | Rₜ = 13.1 min | — |
| | | | 798 [M + H]⁺ | |
| Fmoc-Val-OH | 70 | CDI, 3eq, 2*120 min | Rₜ = 21.4 min | 2% DBU |
| | | | 1119 [M + H]⁺ | |
| | | | 561 [M + 2H]²⁺ | |
| Fmoc-Met-OH | 52 | HATU, 2eq, 60 min | — | 2% DBU |
| | | | | Cleavage |
| | | | | Rₜ = 14.6 min |
| | | | | 1028 [M + H]⁺ |
| | | | | 515 [M + 2H]²⁺ |
| Fmoc-Leu-OH | 49 | HATU, 2eq, 60 min | Rₜ = 22.3 min | 2% DBU |
| | | | 1369 [M + H]⁺ | |
| | | | 683 [M + 2H]²⁺ | |
| Fmoc-Gly-OH | 41 | HATU, 2eq, 60 min | — | 2% DBU |
| Fmoc-Ile-OH | 49 | HATU, 2eq, 60 min | — | 2% DBU |
| Fmoc-Ile-OH | 49 | HATU, 2eq, 60 min | — | 2% DBU |
| Fmoc-Ala-OH•H₂O | 45 | HATU, 2eq, 60 min | — | 2% DBU |
| Fmoc-Gly-OH | 41 | HATU, 2eq, 60 min | — | 2% DBU |
| Fmoc-Lys(Boc)-OH | 65 | HATU, 2eq, 60 min | — | 2% DBU |
| Fmoc-Asn(Trt)-OH | 82 | HATU, 2eq, 60 min | Rₜ = 20.2 min | 2% DBU |
| | | | 1009 [M + 2H]²⁺ | |
| | | | 673 [M + 3H]³⁺ | |
| Boc-Ser(OH)—OH | 28 | PyBOP, 2eq, 60 min | Rₜ = 16.9 min | — |
| | | | 941 [M + 2H]²⁺ | |
| | | | 628 [M + 3H]³⁺ | |
| Fmoc-Gly-OH | 62 | CDI, 3eq, 2*120 min | Rₜ = 19.3 min | 2% DBU |
| | | | 1081 [M + 2H]²⁺ | |
| | | | 721 [M + 3H]³⁺ | |
| | | | 540 [M + 4H]⁴⁺ | |
| Fmoc-Val-OH | 47 | HATU, 2eq, 60 min | — | 2% DBU |
| | | | | Rₜ = 19.1 min |
| | | | | 1019 [M + 2H]²⁺ |
| | | | | 680 [M + 3H]³⁺ |
| Fmoc-Asp(^{t}Bu)-OH | 114 | HATU, 4eq, 60 min | Rₜ = 19.9 min | 2% DBU |
| | | | 1187 [M + 2H]²⁺ | 5% piperidine |
| | | | 792 [M + 3H]³⁺ | |
| | | | 595 [M + 4H]⁴⁺ | |
| Fmoc-Glu(^{t}Bu)-OH | 123 | HATU, 4eq, 60 min | — | 2% DBU |
| | | | | 5% piperidine |
| Fmoc-Ala-OH•H₂O | 90 | HATU, 4eq, 60 min | — | 2% DBU |
| | | | | 5% piperidine |
| Fmoc-Phe-OH | 107 | HATU, 4eq, 60 min | 1362 [M + 2H]²⁺ | 2% DBU |
| | | | 908 [M + 3H]³⁺ | 5% piperidine |
| | | | 681 [M + 4H]⁴⁺ | |
| Fmoc-Phe-OH | 107 | HATU, 4eq, 60 min | — | 2% DBU |
| | | | | 5% piperidine |
| Fmoc-Val-OH | 94 | HATU, 4eq, 60 min | — | 2% DBU |
| | | | | 5% piperidine |
| Fmoc-Leu-OH | 98 | HATU, 4eq, 60 min | 1542 [M + 2H]²⁺ | 2% DBU |
| | | | 1027 [M + 3H]³⁺ | 5% piperidine |
| | | | 771 [M + 4H]⁴⁺ | |
| Fmoc-Lys(Boc)-OH | 130 | HATU, 4eq, 60 min | 1070 [M + 3H]³⁺ | 2% DBU |
| | | | 803 [M + 4H]⁴⁺ | 5% piperidine |
| | | | 643 [M + 5H]⁵⁺ | |
| Fmoc-Gln(Trt)-OH | 169 | HATU, 4eq, 60 min | — | 2% DBU |
| | | | | 5% piperidine |
| Fmoc-His(Trt)-OH | 171 | HATU, 4eq, 60 min | — | 2% DBU |
| | | | | 5% piperidine |
| Fmoc-His(Trt)-OH | 171 | HATU, 4eq, 60 min | 1205 [M + 3H]³⁺ | 2% DBU |
| | | | 903 [M + 4H]⁴⁺ | 5% piperidine |
| | | | 723 [M + 5H]⁵⁺ | |
| | | | Maldi-TOF | |
| | | | 3613 [M + H]⁺ | |
| Fmoc-Val-OH | 94 | HATU, 4eq, 60 min | — | 2% DBU |
| | | | | 5% piperidine |
| Fmoc-Glu(^{t}Bu)-OH | 123 | HATU, 4eq, 60 min | — | 2% DBU |
| | | | | 5% piperidine |
| Fmoc-Tyr(^{t}Bu)-OH | 127 | HATU, 4eq, 60 min | — | 2% DBU |
| | | | | 5% piperidine |
| Fmoc-Gly-OH | 82 | HATU, 4eq, 60 min | 1015 [M + 4H]⁴⁺ | 2% DBU |
| | | | 812 [M + 5H]⁵⁺ | 5% piperidine |
| | | | | Maldi-TOF |
| | | | | 3841 [M + H]⁺ |
| Fmoc-Ser(^{t}Bu)-OH | 106 | HATU, 4eq, 60 min | — | 2% DBU |
| | | | | 5% piperidine |
| Fmoc-Asp(^{t}Bu)-OH | 114 | HATU, 4eq, 60 min | — | 20% piperidine |
| Fmoc-His(Trt)-OH | 171 | HATU, 4eq, 60 min | — | 20% piperidine |
| Fmoc-Arg(Pbf)-OH | 180 | HATU, 4eq, 60 min | Maldi-TOF | 20% piperidine |
| | | | 4554 [M + H]⁺ | |
| Fmoc-Phe-OH | 107 | HATU, 4eq, 60 min | — | 20% piperidine |
| Fmoc-Glu(^{t}Bu)-OH | 123 | HATU, 4eq, 60 min | — | 20% piperidine |
| | | | | Maldi-TOF |
| | | | | 4610 [M + H]⁺ |
| Fmoc-Ala-OH•H₂O | 90 | HATU, 4eq, 60 min | — | 20% piperidine |
| Boc-Asp(^{t}Bu)-OH | 80 | HATU, 4eq, 60 min | — | — |

Solid Phase Synthesis Protocol SP2
Fmoc-Ala-NovaSyn TGA resin 300 mg, loading 0.23 mmol/g

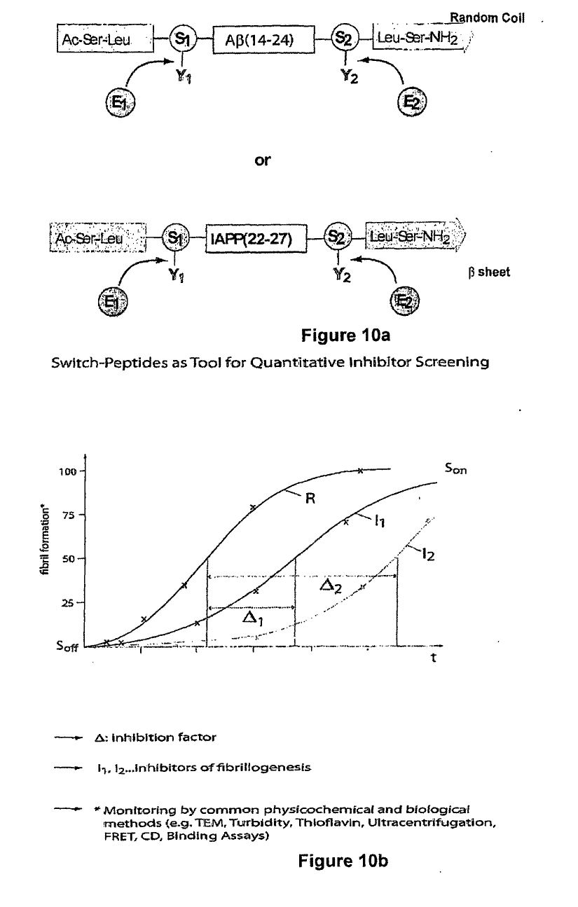

In Vitro Assay of Fibril Formation and Inhibition—Inhibitor Screening System (See FIGS. 10a, 10b)

Amyloid formation was quantitatively evaluated by the fluorescence emission of thioflavin T bound to amyloid fibrils. Aliquots of Aβ (1-42) as control, SP1 and SP2 at concentrations of 0.5 mg/mL prepared in 0.1 M Tris (pH 7.4) were incubated under gentle swirling for 5 days at 37° C. in the presence or absence of enzyme (DPPIV). At the end of the incubation period, 50 mM glycine (pH 9.2) and 2 μM thioflavin T were added in a final volume of 2 mL. Fluorescence was measured at an excitation of 435 nm and an emission of 485 nm in a Perkin Elmer Life Sciences Model LS50B fluorescence spectrometer. Slit widths were set to 5 nm.

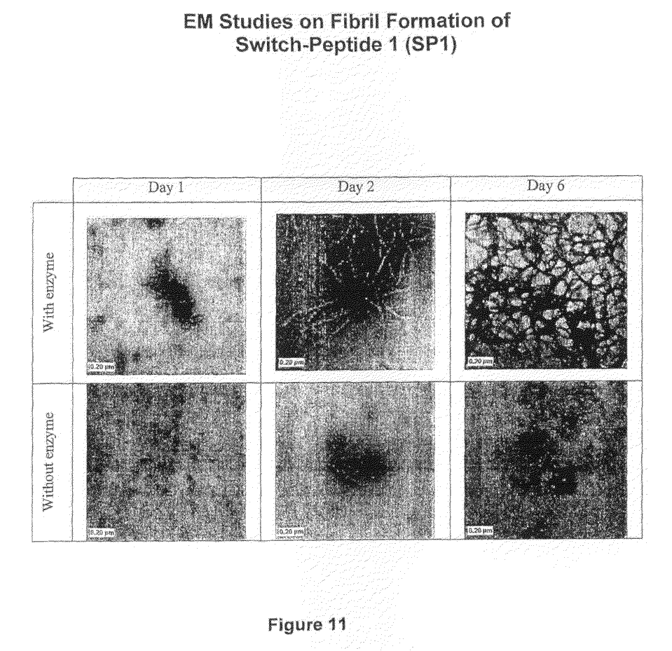

### Electron Microscopy (see FIG. 11)

The incubated samples were centrifuged at 20,000×g for 20 min, and the supernatants were aspirated. The pellets were sonicated for 5 s in 100 μL of water and 8 μL of these suspensions were placed on grids covered by a carbon-stabilized formvar film. Excess fluid was withdrawn after 30 s, and the grids were negatively stained with 3% uranyl acetate in water. The stained grids were then examined and photographed in JEOL 100CX at 60 kV.

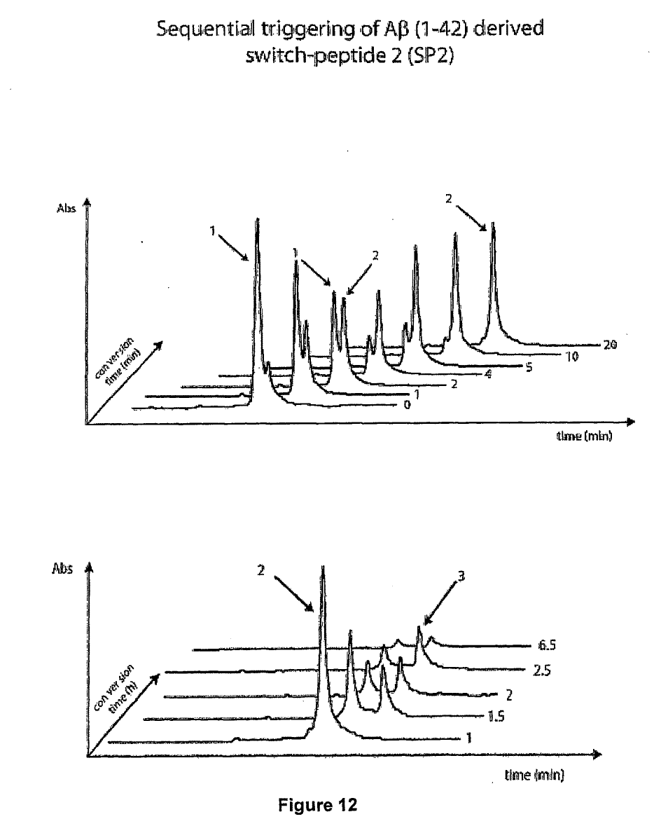

### HPLC Studies of pH- and Enzyme-Triggered Acyl Transfer Migrations on SP2 (FIG. 12)

In switch-peptide 2, S₁ at the N-terminus is triggered by pH. To study the pH-induced acyl transfer, switch-peptide 2 was dissolved in a solution of H₂O/PBS pH 7.4 (9:1) at a concentration of c=0.21 mM. At different times, 10 μL aliquots were removed, quenched by addition of 10 μL HCl 1M and injected to analytical HPLC. FIG. 12 shows the HPLC chromatograms of the chemical conversion of switch-peptide 2. These chromatograms show two different peaks: peak 1 at Rₜ=17.4 mM corresponds to the S_{off} state and peak 2 at Rₜ=17.6 mM represents compound SP2 where the acyl migration at switch S₁ occurred restoring native Aβ(1-36). Gradually, the absorbance of peak 1 decreases and the absorbance of peak 2 increases. pH-induced acyl migration proceeds very fast, and within 20 minutes, the acyl migration at S₁ is complete. No precipitation was associated with the activation of S₁ suggesting that Aβ(1-36) has no tendency for aggregation. We then added to the solution 6.5 μg (6000-fold excess of substrate) of DPPIV to induce switch S₂. At different times, 20 μL aliquots were removed and injected to analytical HPLC. FIG. 12 shows the HPLC chromatograms of the enzyme-triggered acyl migration of switch-peptide 2. These chromatograms show two different peaks: peak 2 at Rₜ=17.6 min corresponds to the activated switch S₁ and peak 3 at Rₜ=18.2 min represents switch-peptide 2 where both switches are in the Sₒₙ state. Gradually, the absorbance of peak 2 decreases and the absorbance of peak 3 increases. Interestingly, by the subsequent enzymatic switching on of the C-terminal segment (37-42), the characteristic phenomena observed for native Aβ(1-42) i.e. aggregation and precipitation are initiated (drastic decrease in absorbance of peak 3 and complete disappearance after a few hours).

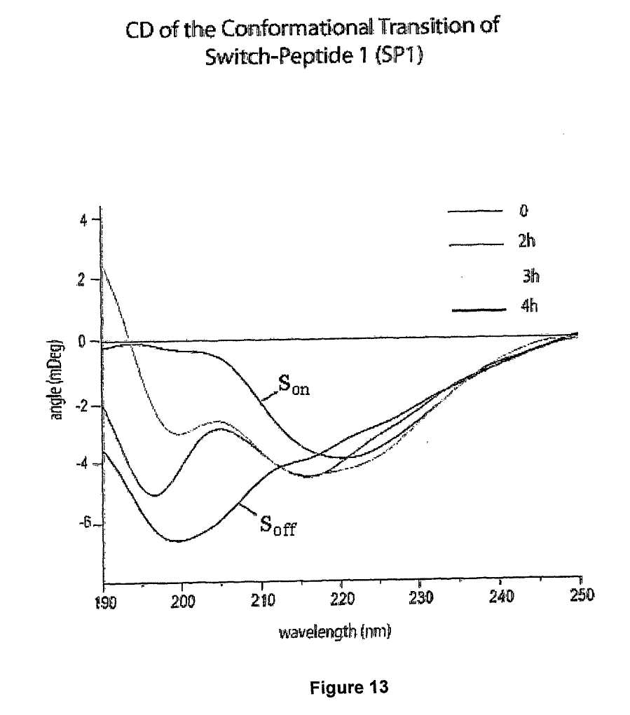

### CD Studies (FIG. 13)

To study the conformational transition of switch-peptide 2, the peptide was dissolved in a solution of H₂O/PBS pH 7.0/TFE 60:35:5 (c=21 μM) and DPPIV was added. At time zero the CD spectrum was characteristic of a random coil with a strong negative Cotton effect at λ=198 nm (black curve). After 4 hours, this negative Cotton effect disappeared and a negative Cotton effect at =218 nm characteristic of a β-sheet appeared (FIG. 13).

The activation of both switches induced a conformational transition of the amyloid sequence from random coil (S_{off}) to β-sheet (Sₒₙ). The same experiment was carried out without the enzyme to investigate the influence of each switch independently. When S₂ was off, the negative Cotton effect at λ=198 nm did not disappear, and after 4 h the spectrum was still characteristic of a random coil. The C-terminus seems essential for β-sheet formation; insertion of the switch in this region prevents aggregation and β-sheet formation. This suggests that initial interactions in the amyloid β sequence or between β-structure peptides involve first this hydrophobic C-terminal end (37-42). These results concur with the conformational studies of Barrow and Zagorski, who have shown that Aβ(1-42) forms more stable β-sheet structures than Aβ(1-39). A recent study on the conformational transition of amyloid β-peptide have revealed the importance of the four glycines (Gly²⁵, GlY²⁹, GlY³³ and GlY³⁷) for Aβ(1-40) to form β-sheets in aqueous solution. Mutations of these glycines to alanines almost abolished the β-sheet formation and increased the content of the helix component. Applicant demonstrated that one of the glycines (Gly³⁷) can be substituted in Aβ(1-42) without disruption of β-sheet formation.

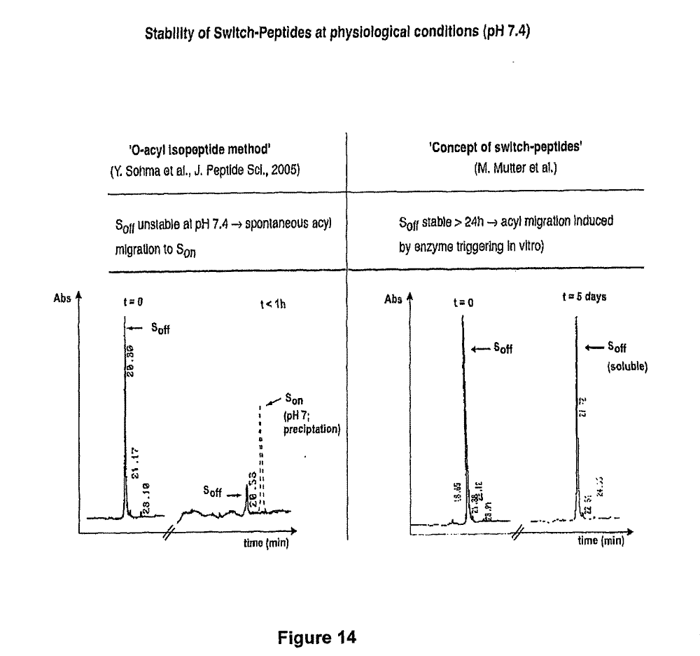

### Stability of Switch-Peptides (FIG. 14)

Before studying enzyme-triggered acyl migration of switch peptides, the properties of switch-peptides in the S_{off} state were explored. High stability of switch-peptides in PBS, pH 7.4, at room temperature was demonstrated (FIG. 12). No hydrolysis of the ester bond occurred and the peptide remains soluble indicating that acyl migration was prevented due to the presence and stability of the enzyme-cleavable protection (e.g. dipeptides: Axx-Pro, nonproteinogenic amino acids: D-Axx, pyroGlu etc.).

### Enzyme-Triggered Acyl Migrations (Table 3 and 4)

In the first generation of switch-peptides we used the DPP family as enzyme for triggering acyl migrations (Table 3).

TABLE 3
Enzyme family DPPIV used for enzyme-triggered acyl migration.

| Y | Enzyme |
|---|---|
| Xaa-Pro (e.g. Arg-Pro, | DPPIV |
| Orn-Pro, Lys-Pro, Val-Pro) | |
| pGlu | Pyroglutamate aminopeptidase |
| PhAc | Penicillin amidase |
| Ac | Acylase |
| D-amino acids | D-Aminopeptidase |

In the second generation of switch-peptides, we introduced enzyme labile groups as Y protection for triggering O→N migrations. To this end, hydrolases (EC 3) acting on non-peptidic peptidic bonds (EC 3.4) or peptidic C—N bonds (EC 3.5) were investigated. Specifically, acylase cleaving an acetyl group, penicillin G amidase a phenylacetyl group, pyroglutamate aminopeptidase a pyroglutamic amino acid and dipeptidyl aminopeptidase a Axx-Pro (for example Arg-Pro) dipeptide were applied.

Due to electronic or steric factors, the potentially enzyme labile groups of switch-peptides were not cleaved by acylase and penicillin amidase. To spatially separate the enzyme cleavable group Y from the backbone of the switch-peptide, flexible linkers were introduced (Table 4).

TABLE 4
Different types of linker systems used for enzymatic triggering of acyl migrations.

| Y | Linker | Enzyme |
|---|---|---|
| Acoeoc | 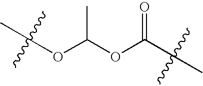 | Esterase |
| AcOZ | 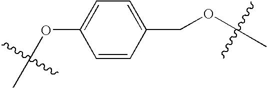 | Esterase |
| PhacOZ | 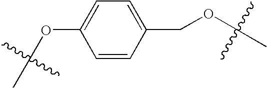 | Penicillin amidase |

As a consequence, bulky amino acid side chains in the target peptide will not limit the steric accessibility of the enzyme. The resulting switch-peptides are composed by three main units: a protecting group Y as enzyme substrate, a chemically labile linker that undergoes spontaneous fragmentation upon cleavage of the enzyme-sensitive bond and a target switch-peptide.

### Systematic Procedure for Screening Inhibitors

### General Remarks

Kinetic studies of Aβ fibrillogenesis are complicated. This is because of the fact that normally fibril formation occurs very rapidly and sensitive to the method of preparation of stock solutions [24]. CD monitoring of the stock solutions of Aβ 40 showed a slow formation of β-sheet over a period of one day [25]. Similarly, amylin peptide also is reported to rapidly aggregate and form fibrils in water [26].

Fluorinated alcohols, like 2,2,2-trifluoroethanol (TFE) and hexafluoro isopropanol (HFIP), are known to denature native protein structure [27] and disturb intermolecular self-assembly processes of amyloid-forming peptides. Therefore, HFIP has recently found increasing applications as a solvent for handling amyloid-forming peptides [28].

To make Applicant's screening system practically useful, the absence of any nucleation seeds in the CD solutions at the beginning of the experiments was essential. Therefore, stock solutions of completely non-aggregated peptides were required. This is because the quality of the kinetic data obtained was found to depend on the age of the stock solution. The most reproducible data were obtained when the solution was prepared immediately before the experiment. But, weighing the solid sample each time also generated a certain error. To get rid of these experimental problems, a general method of sample preparation was established and applied to all the experiments.

### Sample Preparation

At first, a calculated amount (weight) of the peptides was dissolved in spectrophotometric grade hexafluoro isopropanol. This solution was divided into several aliquots of a specific volume in Eppendorfs. Three times by volume of water was added to each of them and then lyophilized. The lyophilized samples were kept in −18° C. for future use. Theses samples were taken out of the refrigerator just before use, and redissolved in calculated amounts of spectrophotometric grade MeOH. A specific volume (aliquot) of this solution was used for a single experiment. The starting conformational state of switch-peptides under these conditions was highly reproducible and allowed us to follow kinetics of the onset of beta sheet structure and peptide aggregation. Therefore, this method was followed for the screening system of β-breakers.

Agitation of several soluble proteins, such as Sup35 [9], insulin [30], and variants of the B1 in of streptococcal IgG-binding protein G(β1) [31], has been shown to accelerate fibril formation in vitro. The cause of agitation induced fibril formation is not clear. However, it is believed that agitation may cause shearing of the first-formed fibrils, thereby increasing the number of nucleation particles (seeds) and accelerating fibril formation²⁹¹. Alternatively, it is believed that agitation may increase the apparent concentration of protein by removing the amount of protein that sticks to the walls of the tubes. However, fibrils do form in the absence of agitation, only more slowly.

As it is reported that agitation influences on fibril formation, a fixed time of vortex and sonication was applied for mixing the peptides for each experiment of the β-breaker screening procedure in order to get a reproducible result.

Experimental Procedure (pH-Triggered HGSP)

The overall procedure of screening system is summarized below.

1) 1 mg of each Host-guest switch-peptide (HGSP) and inhibitor (e.g. peptide-based β-breakers) was dissolved in 1 ml of HFIP in an Eppendorf.
2) Aliquots of 50 μL were separated in several Eppendorfs. 150 μL of bidistilled water was added to each of them and lyophilized. Lyophilized samples can be kept in deep freezer for several weeks.
3) To start the experiment, the sample was redissolved in 50 μL of methanol and used immediately. To dissolve the peptide properly, 30 seconds of vortex and 30 seconds of sonication were applied in each case.
A specific volume (10 or 20 μL) of the switch-peptide and n-breaker solution was taken in another Eppendorf for incubation. Total volume of methanol did not exceed 50 μL. 100 μL of phosphate-citrate buffer (pH 3.6, c=1 mM) was added to keep the switch-peptide in S_{off} state during incubation. Total volume was maintained to be 980 μL in each cases, amount of DDW was changed with respect to the total volume of the peptide and inhibitor mixture. 30 seconds of vortex was applied in order to mix the peptides properly, and kept for incubation 15 minutes.
5. After the incubation, 20 μL of PBS (pH 7.1, C=0.15 M) were added to trigger the switch for acyl migration, and the solution mixture were transferred to a 1 ml quartz cuvette (path length 1 cm).
6. Interval scan measurement was performed during 2 h or 5 h in 2 minutes or 5 minutes interval respectively depending on the experiment. Each experiment was repeated 3 times.
7. The spectra obtained at different times was subtracted by the spectrum obtained at t=0, to get the difference spectra. Then the ellipticity values were plotted against time. This indicates the amount of β-sheet evolved in the solution with time.
8. Average curve of three consecutive experiments was fitted by Boltzman equation and lag time, half life time and apparent rate constant of aggregation were calculated. Lag, half life inhibition, % inhibition and growth inhibition were also calculated. All arithmetic operations with standard deviations used in this study are calculated using the software provided by John C. Pezzullo, in his Interactive Statistics Page, where well known standard methods of propagation of standard errors for arithmetic operations were used. (URL address: http://members.aol.com/iohnp71/erpropgt.html). Error correlations between the variables were taken zero in each case where two variables were involved.

### REFERENCES

[1] Self-assembling Peptide-Systems in Biology, Medicine and Engineering (Eds.: A. Aggeli, N. Boden, S. Zhang), Kluwer Acad. Publ. Dordrecht, 2001 and references therein.
[2] Selkoe, D. J. Physiol. Rev. 2001, 81, 741-766.
[3] Bernstein, S. L.; Wyttenbach, T.; Baumketner, A.; Shea, J.-A.; Bitan, G.; Teplow, D. B.; Bowers, M. T. J. Am. Chem. Soc. 2005, 127, 2075-2084.
[4] Soto, C. Nature Rev. Neurosci. 2003, 4, 49-60.
[5] Gorman, P. M.; Chakrabartty, A. Biopolymers (Peptide Sci.) 2001, 60, 381-394.
[6] Toniolo, C.; Bonora, G. M.; Mutter, M. J. Am. Chem. Soc. 1979, 101, 450-454.
[7] Bosques, C. J.; Imperiali, B. J. Am. Chem. Soc. 2003, 125, 7530-7531.
[8] Mutter, M.; Gassmann, R.; Buttkus, U.; Altmann, K.-H. Angew. Chem. Int. Ed. 1991, 30, 1514-1516.
[9] a) Mutter, M.; Chandravarkar, A.; Boyat, C.; Lopez, J.; Dos Santos, S.; Mandal, B.; Mimna, R.; Murat, K.; Patiny, L.; Saucède, L.; Tuchscherer, G. Angew. Chem. Int. Ed. 2004, 43, 4172-4178; b) Dos Santos, S.; Chandravarkar, A.; Mandal, B.; Mimna, R.; Murat, K.; Saucede, L.; Tella, P.; Tuchscherer, G.; Mutter, M. J. Am. Chem. 2005, 127, 11888-11889.
[10] Shao, Y.; Paulus, H. J Peptide Res. 1997, 50, 193-198.
[11] Coltart, D. M. Tetrahedron 2000, 56, 3449-3491.
[12] Hamada, Y.; Matsumoto, H.; Yamaguchi, S.; Kimura, T.; Hayashi, Y.; Kiso, Y. Bioorg. Med. Chem. 2002, 10, 4155-4167.
[13] Carpino. L. A.; Krause, E.; Sferdean, C. D.; Schühmann, M.; Fabian, H.; Bienert, M.; Beyermann, M. Tetrahedron Lett. 2004, 45, 7519-7523.
[14] a) Sohma, Y.; Sasaki, M.; Hayashi, Y.; Kimura, T.; Kiso, Y. Tetrahedron Lett. 2004, 45, 5965-5968; b) Sohma, Y.; Hayashi, Y.; Kimura, M.; Chiyomori, Y.; Taniguchi, A.; Sasaki, M.; Kimura, T.; Kiso, Y. J Peptide Sci. 2005, 11, 441-451.
[15] Houben-Weyl, Methods of Organic Chemistry (Goodman, M.; Felix, A.; Moroder, L.; Toniolo, C., Eds), Vol. E 22d, Thieme: Stuttgart: 2003.
[16] Shepard, N. E.; Abbenante, G.; Fairlie, D. P. Angew. Chem. Int. Ed., 2004, 43, 2687-2690.
[17] Dos Santos, S.; Chandravarkar, A.; Lashuel, H.; Mutter, M., in prep.
[18] Adessi, C.; Frossard, M.-J.; Boissard, C.; Fraga, S.; Bieler, S.; Rueckle, T.; Vilbois, F.; Robinson, S. M.; Mutter, M.; Banks, W. A.; Soto, C. J. Biol. Chem. 2003, 278, 13905-13911.
[19] Filip, S. V.; Cavelier, F. J. Peptide Sci. 2004, 10, 115-118.
[20] a) J. M. Stewart, J. D. Young, Solid Phase Peptide Synthesis, Pierce Chemical, Rockford, Ill., 1984; b) G. A. Grant, Synthetic Peptides (2^{nd} ed.) 2002, Oxford University Press, Inc.
[21]: Esler, W. P.; Stimson, E. R.; Ghilardi, J. R.; Vinters, H. V.; Lee, J. P.; Mantyh, P. W.; Maggio, J. E. Biochemistry 1996, 35, 749-757.
[22] Esler, W. P.; Stimson, E. R.; Ghilardi, J. R.; Felix, A. M.; Lu, Y.-A.; Vinters, H. V.; Mantyh, P. W.; Maggio, J. E. Nature Biotechnology 1997, 15, 258-262.
[23] Findeis, M. A.; Musso, G. M.; Arico-Muendel, C. C.; Benjamin, H. W.; Hundal, A. M.; Lee, J.-J.; Chin, J.; Kelley, M.; Wakefield, J.; Hayward, N. J.; Molineaux, S. M., Polymerization. Biochemistry 1999, 38, 6791-6800.
[24] Snyder, S.; Ladror, U.; Wade, W.; Wang, G.; Barrett, L., et al. Biophys. J. 1994, 67, 1216-28.
[25] Wilkins, D. K.; Dobson, C. M.; Groβ, M. Eur. J. Biochem. 2000, 267, 2609-2616.
[26] Kapurniotu, A.; Bernhagen, J.; Greenfield, N.; Al-Abed, Y.; Teichberg, S.; Frank, R. W.; Voelter, W.; Bucala, R. European Journal of Biochemistry 1998, 251, (1-2), 208-216.
[27] Hirota, N.; Mizuno, K.; Goto, Y. J. Mol. Biol. 1998, 275, 365-378.
[28] Wood, S. J.; Maleeff, B.; Hart, T.; Wetzela, R. Journal of Molecular Biology 1996, 256, (5), 870-877.
[29] Serio, T. R.; Cashikar, A. G.; Kowal, A. S.; Sawicki, G. J.; Moslehi, J. J.; Serpell, L.; Amsdorf, M. F.; Lindquist, S. L. Science 2000, 289, (5483), 1317-1321.
[30] Nielsen, L.; Khurana, R.; Coats, A.; Frokjaer, S.; Brange, J.; Vyas, S.; Uversky, V. N.; Fink, A. L. Biochemistry 2001, 40, (20), 6036-6046.
[31] Ramirez-Alvarado, M.; Merkel, J. S.; Regan, L. Natl Acad Sci U S A. 2000, 97, (16), 8979-8984.

1. A method for the manufacture of a peptidic folding precursor (Switch-Peptide), stable and soluble at physiological conditions, derived from a peptide having a potential for self-assembling and fibrillogenesis, wherein said method comprises the steps of:
generating at least one Switch Element (S-element) in the amino acid sequence of said peptide, characterized in that said Switch Element having the formula:
H₂N-Axx-Pro-CO—
wherein Axx represents any proteinogenic or non proteinogenic amino acid residue and,
subsequently reacting said modified peptide with an enzyme of the DPP IV family specifically triggering O,N-acyl migration, wherein said enzyme is selected for its specificity toward said Switch Element.

2. The method of claim 1, wherein Axx represents a basic or a hydrophobic amino acid residue.

3. The method of claim 1, wherein the enzyme of the DPP IV family having specificity toward the Switch Element binding site is selected among the group comprising DPP IV, Esterase, Acylase, Phenacylase, Penicillin G amidase, D-aminopeptidase.

4. The method according to claim 1, wherein the Switch Element is generated on at least one of a Serine, Cysteine, Threonine and on any other conservative replacements by Ser, Cys, Thr in the native amino acid sequence of said peptide.

5. The method of claim 1, wherein the peptide having a potential for self-assembling and fibrillogenesis is able at least one of to adopt a beta-pleated sheet conformation and to form oligomers, fibrils and plaques.

6. The method of claim 5, wherein the peptide having a potential for self-assembling and fibrillogenesis is Amyloid beta, alpha synuclein, Huntingtin, Islet Amyloid protein or prion Proteins.

7. A tool for the quantitative, controlled in vitro study of fibrillogenesis and its inhibition of peptides involved in degenerative diseases comprising:
generating multi-switch elements in a peptide sequence of a peptide able to form fibrillar aggregates, wherein said multi-switch elements comprise at least one Switch Element having the formula:
H₂N-Axx-Pro-CO—
wherein Axx represents any proteinogenic or non proteinogenic amino acid residue and,
switching on selectively, independently and orthogonaly said multi-switch elements one by one, by reacting said peptide with an enzyme of the DPP IV family specifically triggering O,N-acyl migration, wherein said enzyme is selected for its specificity toward said Switch Element,
monitoring the resulting self-assembling, oligomerisation, aggregation and fibrillogenesis by at least one of analytical, spectroscopic or biophysical methods and in vitro tests.

8. The tool of claim 7, wherein multi-switch elements comprise at least two switch elements inserted:
a) in the middle of the guest sequence or/and
b) at the C- or N-terminal end of the guest sequence.

9. The tool of claim 7, wherein the enzyme of the DPP IV family is selected among the group comprising DPP IV, Esterase, Acylase, Phenacylase, Penicillin G amidase, D-aminopeptidase.

10. An in vitro system for the screening of the inhibitory effect of β-breaker or fibril disrupting molecules acting on peptides able to form fibrillar aggregates and involved in degenerative diseases comprising:
a) generating a modified peptide containing one or two switch-elements (Soff-state) comprising at least one Switch Element having the formula:
H₂N-Axx-Pro-CO—
wherein Axx represents any proteinogenic or non proteinogenic amino acid residue and,
comprising a fibril nucleation sequence (guest) and a beta-sheet promoting host;
b) adding a potential of at least one of a β-sheet inhibitor and fibril disrupting molecule or libraries thereof and,
monitoring the resulting self-assembling, oligomerisation, aggregation and fibrillogenesis after at least one of chemical and enzymatic triggering of acyl migration (Son) by at least one of analytical, spectroscopic or biophysical methods and in vitro tests.

11. The in vitro system of claim 10, wherein peptides able to form fibrillar aggregates and involved in degenerative diseases are selected among Amyloid beta, alpha synuclein, Huntingtin, Islet Amyloid protein or prion Proteins and transformed to host-guest switch-peptides according to claim 10.

12. A stable and soluble peptidic folding precursor (Switch Peptide) derived from a peptide having a potential for self-assembling and fibrillogenesis, obtained by the method according to claim 1.

13. The stable and soluble peptidic folding precursor (Switch Peptide) of claim 12, wherein the peptide having a potential for self-assembling and fibrillogenesis is Amyloid beta, alpha synuclein, Huntingtin, Islet Amyloid protein or prion Proteins.

14. The method of claim 2, wherein the enzyme of the DPP IV family having specificity toward the Switch Element binding site is selected among the group comprising DPP IV, Esterase, Acylase, Phenacylase, Penicillin G amidase, D-aminopeptidase.

15. The method according to claim 2, wherein the Switch Element is generated on at least one of a Serine, Cysteine, Threonine and on any other conservative replacements by Ser, Cys, Thr in the native amino acid sequence of said peptide.

16. The method according to claim 3, wherein the Switch Element is generated on at least one of a Serine, Cysteine, Threonine and on any other conservative replacements by Ser, Cys, Thr in the native amino acid sequence of said peptide.

17. The method of claim 2, wherein the peptide having a potential for self-assembling and fibrillogenesis is able to adopt at least one of a beta-pleated sheet conformation and to form oligomers, fibrils and plaques.

18. The method of claim 3, wherein the peptide having a potential for self-assembling and fibrillogenesis is able to adopt at least one of a beta-pleated sheet conformation and to form oligomers, fibrils and plaques.

19. The method of claim 4, wherein the peptide having a potential for self-assembling and fibrillogenesis is able to adopt at least one of a beta-pleated sheet conformation and to form oligomers, fibrils and plaques.

20. The tool of claim 8, wherein the enzyme of the DPP IV family is selected among the group comprising DPP IV, Esterase, Acylase, Phenacylase, Penicillin G amidase, D-aminopeptidase.
